(19) United States
(12) Patent Application Publication
(10) Pub. No.: US 2009/0024021 A1
(43) Pub. Date: Jan. 22, 2009

(54) Methods and Systems for Using Transcranial Magnetic Stimulation and Functional Brain Mapping for Examining Cortical Sensitivity, Brain Communication, and Effects of Medication

(76) Inventors: Mark S. George, Sullivan's Island, SC (US); Daryl E. Bohning, Warren, CT (US); Ziad Nahas, Charleston, SC (US)

Correspondence Address: Ballard Spahr Andrews & Ingersoll, LLP, SUITE 1000, 999 PEACHTREE STREET, ATLANTA, GA, 30309-3915, US

(21) Appl. No.: 12/035,997

(22) Filed: Feb. 22, 2008

Related U.S. Application Data
(63) Continuation of application No. 10/535,775, filed on Mar. 8, 2006, now abandoned.
(60) Provisional application No. 60/431,820, filed on Dec. 9, 2002.

(51) Int. Cl. A61B 5/055; A61N 2/00

(52) U.S. Cl. 600/411; 600/13

(57) ABSTRACT

Transcranial magnetic stimulation is interleaved with functional brain imaging to examine cortex sensitivity and brain communication and to determine efficacy of medication.

### CROSS-REFERENCE TO RELATED APPLICATIONS

This application claims priority to U.S. Provisional Applications No. 60/377,692 and No. 60/431,820, herein incorporated by reference.

### BACKGROUND

The present invention generally relates to the use of transcranial magnetic stimulation in conjunction with functional magnetic resonance imaging. More particularly, the present invention relates to the use of transcranial magnetic stimulation (TMS) interleaved with fMRI to measure cortical sensitivity, brain communication, and to determine efficacy of medications, such as central nervous system active compounds.

For over a century, it has been recognized that electricity and magnetism are interdependent (Maxwell's equations) (Bohning, 2000). Passing current through a coil of wire generates a magnetic field perpendicular to the current flow in the coil. If a conducting medium, such as the brain, is adjacent to the magnetic field, current will be induced in the conducting medium. The flow of the induced current will be parallel, but opposite in direction, to the current in the coil (Cohen et al., 1990; Brasil-Neto et al., 1992; Saypol et al., 1991; Roth et al., 1991). Thus, transcranial magnetic stimulation (hereinafter “TMS”) has been referred to as “electrodeless” electrical stimulation to emphasize that the magnetic field acts as the medium between electricity in the coil and induced electrical currents in the brain.

TMS involves placing an electromagnetic coil on the scalp. Subjects are awake and alert. There is some discomfort, in proportion to the muscles that are under the coil, and to the intensity and frequency of stimulation. Subjects usually notice no adverse effects except for occasional mild headache and discomfort at the site of the stimulation. High intensity current is rapidly turned on and off in the coil through the discharge of capacitors. This produces a time-varying magnetic field that lasts for about 100-300 microseconds. The magnetic field typically has a strength of about 2 Tesla (or 40,000 times the earth's magnetic field, or about the same intensity as the static magnetic field used in clinical MRI). The proximity of the brain to the time-varying magnetic field results in current flow in neural tissue.

The technological advances made in the last 15 years led to the development of magnetic stimulators that produce sufficient current in brain to result in neuronal depolarization.

Neuronal depolarization can also be produced by electrical stimulation, with electrodes placed on the scalp (referred to as transcranial electric stimulation (“TES”)). Importantly, unlike electrical stimulation, where the skull acts as a massive resistor, magnetic fields are not deflected or attenuated by intervening tissue. This means that TMS can be more focal than TES. Furthermore, for electrical stimulation to achieve sufficient current density in brain to result in neuronal depolarization, pain receptors in the scalp must be stimulated (Saypol et al., 1991).

A striking effect of TMS occurs when one places the coil on the scalp over primary motor cortex. A single TMS pulse of sufficient intensity causes involuntary movement. The magnetic field intensity needed to produce motor movement varies considerably across individuals and is known as the motor threshold (Kozel et al., 2000; Pridmore et al., 1998). Placing the coil over different areas of the motor cortex causes contralateral movement in different distal muscles, corresponding to the well-known homunculus. TMS can be used to map the representation of body parts in the motor cortex on an individual basis. Subjectively, this stimulation feels much like a tendon reflex movement. Thus, a TMS pulse produces a powerful but brief magnetic field which passes through the skin, soft tissue, and skull and induces electrical current in neurons, causing depolarization which then has behavioral effects (body movement).

Single TMS over the motor cortex can produce simple movements. Over the primary visual cortex, TMS can produce the perception of flashes of light or phosphenes (Amassian et al., 1995). To date, these are the ‘positive’ behavioral effects of single pulse TMS. Other immediate behavioral effects are generally disruptive. Interference with, and perhaps augmentation of, information processing and behavior is especially likely when TMS pulses are delivered rapidly and repetitively. Repeated rhythmic TMS is called repetitive TMS (rTMS). If the stimulation occurs faster than once per second (1 Hz), it is modified as fast rTMS.

rTMS at frequencies of around 1 Hz has been shown to produce inhibition of the motor cortex. rTMS at higher frequencies of several minutes has been shown to excite underlying cortex for several minutes. Manipulations of frequency and intensity may produce distinct patterns of facilitation (fast rTMS) and inhibition (slow rTMS) of motor responses with distinct time courses. These effects may last beyond the duration of the rTMS trains with enduring effects on spontaneous neuronal firing rates. Determining whether, in fact, lasting increases and decreases in cortical excitability can be produced as a function of rTMS parameters, and whether such effects can be obtained in areas outside of the motor cortex, are of key importance.

TMS is generally safe with no side effects except mild headache in about 5% of subjects. Higher frequency TMS can produce seizures. With the publication of safety tables in 1998, there have been no unintended seizures produced in the world (Wassermann et al., 1996b; Wassermann, 1997; Wassermann et al., 1996a). Animal studies, along with human post-mortem and brain imaging studies (Nahas et al., 2000a), have all failed to find any pathological effects of TMS (Lorberbaum & Wassermann, 2000).

TMS evoked motor responses result from direct excitation of corticospinal neurons at or close to the axon hillock. It is thought that the TMS magnetic field induces an electrical current in the superficial cortex. The TMS magnetic field declines exponentially with distance from the coil. This limits the area of depolarization with current technology to a depth of about 2-cm below the brain's surface. Nerve fibers that are parallel to the TMS coil (perpendicular to the magnetic field) are more likely to depolarize than those perpendicular to the coil. It is thought, as well, that bending nerve fibers are more susceptible to TMS effects than straight fibers (Amassian et al., 1995.

Conventional TMS coils are either round or in the shape of a figure eight (Cohen et al., 1990). The figure eight designs are more focal than the round coils. Most coils are mere copper wire either alone or wrapped around a solid metal core. Because most coils are inefficient, they produce heat as a byproduct. The solid coils are more efficient, without a heating problem. Other manufacturers have used water cooling (Cadwell), or air cooling (Magstim) to deal with this issue. DARPA materials science research might drastically improve the current technology.

The peak effect of TMS can be localized to within less than a millimeter in terms of functional location. More work is needed in terms of actually understanding the exact location of TMS effects (Bohning et al., 2001; Bohning et al., 1997). There is much debate about whether one could devise an array of coils in such a way as to stimulate deep in the brain without overwhelming the superficial cortex.

Since it was first developed (1 (Citation List 1)), TMS has been used to test nerve connections (2-6), nerve excitability (7-9), and nerve conduction times (10) in peripheral nerves (for review, see Ref. 11). One might think of this as testing a circuit with two anatomically separate active areas with a single connection. Paus et al (12) demonstrated that TMS might be combined with neuroimaging to explore the connectivity of more complex three dimensional networks in the brain, allowing the direct assessment of neural connectivity without requiring the subject to engage in any specific behavior.

Recently, TMS interleaved with functional neuroimaging has been successfully implemented by a small but growing number of research groups. In 1997, the first TMS/PET results were reported by Paus et al (12) and Fox et al (13). Also, in 1997, Ilmoniemi et al. (14) reported the first success with TMS/EEG. In 1998, Bohning et al (15) described the first successful interleaving of TMS and fMRI at 1.5 T. Interleaved TMS/fMRI has been shown to be effective in applications such as deception detection and inhibition, as described, e.g., in commonly assigned U.S. Provisional Application No. 60/341,297 filed Dec. 13, 2001 entitled “System and Method of Detecting Deception by fMRI,” U.S. Provisional Patent Application No. 60/396,054 filed 15 Jul. 2002 entitled “Functional Magnetic Resonance Imaging Guided Transcranial Magnetic Stimulation Deception Inhibitors,” and U.S. Provisional Patent Application Ser. No. 60/341,137 filed Dec. 13, 2001 entitled “fMRI-Compatible Skin Conductance Response (SCR) Monitor”, and PCT Application No. PCT/US02/40142 filed Dec. 13, 2002. These applications are incorporated herein in their entirety by this reference.

fMRI has better spatial and temporal resolution than PET, and because it does not use ionizing radiation, it is more suitable for repeated and long-term studies. It is also readily available, with 1.5 T MR scanners installed in medical centers around the world. Hence, TMS/fMRI is potentially the most promising of the three.

Despite all the advances made using TMS/fMRI, there still exists a need for a technique and system for adequately examining brain communication and cortical sensitivity. There also exists a need for examining effects of medication on the brain.

### SUMMARY

According to exemplary embodiments, methods and systems are provided for using transcranial magnetic stimulation in conjunction with functional magnetic resonance imaging to open up a whole new area of noninvasive in-vivo research into brain cortex excitability and connectivity and an objective means for applying and measuring the efficacy of therapeutic intervention.

According to a first aspect, the TMS paired-pulse technique can be combined with BOLD-fMRI neuroimaging, both for testing cortical sensitivity in areas other than motor cortex, and for using the BOLD response amplitude dependence on TMS ISI to investigate brain communication at high time resolution.

According to another aspect, interleaved TMS/fMRI may be used to examine medication effects (a process we now refer to as interleaved TMS/pharmacological MRI-phMRI).

These and other aspects will become apparent from the following description of various embodiments taken in conjunction with the Appendices, although variations and modifications may be effected without departing from the spirit and scope of the novel concepts of the disclosure.

### BRIEF DESCRIPTION OF THE DRAWINGS

FIG. 1 illustrates shows a schematic of an exemplary TMS/fMRI setup;

FIG. 2 illustrates an exemplary schematic for TMS coil holder/head positioner;

FIG. 3 illustrates an exemplary MR-guided TMS Coil Holder showing degrees of freedom;

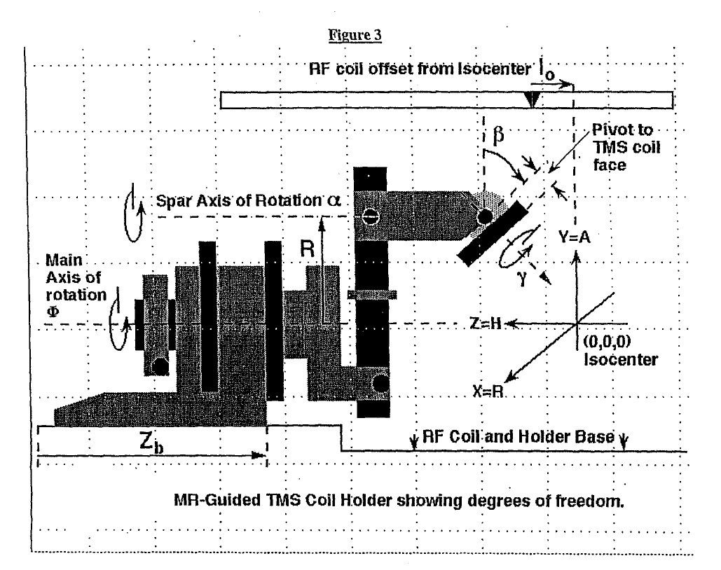

FIGS. 4A and 4B graphically illustrate BOLD time course with model fit for the ipsi-lateral motor cortex and the contra-lateral auditory cortex, respectively;

FIGS. 5A and 5B are graphs of the amplitude scaling factor vs. ISI for the ipsi-lateral motor cortex and the contra-lateral auditory cortex, respectively;

FIG. 6 depicts a subject individual being positioned for functional brain imaging using an MRI scanner;

FIG. 7 depicts a subject individual with a TMS system including a translational/positioning system;

FIG. 8 depicts a block diagram of an exemplary study design for a study conducted in accordance with exemplary embodiments;

FIG. 9 illustrates relative timing of a cycle of interleaved TMS and fMRI scanning in an exemplary study. One cycle consists of six 21-sec subcycles, four rest and two TMS. During each subcycle, the scanner acquires seven sets of 15 transverse images. Each subject received two interleaved TMS/phMRI scans each visit, one using TMS over the left motor cortex and the second run with TMS over the left prefrontal cortex.

FIG. 10 graphs regions of activation during TMS over the motor cortex. TMS resting motor threshold data for all 12 subjects showed a significant increase on the day that subjects received LTG, compared with placebo. (Student's t-test, t=3.41, p<0.01) FIGS. 11A and 11B illustrate brain images taken during an exemplary study under various conditions of the study. These are the group data in 10 subjects for Motor Cortex and 8 subjects for Prefrontal Cortex stimulation. The group differences of TMS-Rest are shown depicted on a representative brain in Talairach coordinates. On the left of the image are the results for TMS over Motor Cortex stimulation at 120% RMT for Placebo (top), LTG (middle), and the difference between LTG and placebo (bottom) (all contrasts, p<0.001, extent 0.05). Note that motor cortex TMS causes local and distant activation, and that LTG reduced this TMS induced activity both locally under the coil and in connected regions. On the right of the image are the results for TMS over Prefrontal Cortex stimulation at 100% RMT for Placebo (top), LTG (middle), and the difference between LTG and placebo (bottom) (all contrasts, p<0.001, extent 0.05). Note that prefrontal cortex TMS causes limbic system activation, and that LTG increases this activity. The LTG induced increases (on the bottom right panel) are depicted at a lower statistical threshold than the other results (p<0.05). M1=Motor cortex, Hi=Hippocampus, AA=Auditory area, OFC=Orbitofrontal cortex.

FIGS. 12A and 12B provide graphs showing number of active voxels by study subject for motor cortex stimulation and prefrontal cortex stimulation. The number of significant voxels in individuals in a region of interest directly underneath the TMS coil, during Motor Cortex stimulation (120% RMT minus rest over motor cortex) for the LTG day and the placebo day. Compared with placebo, LTG significantly decreased the number of active voxels in the motor cortex (Wilcoxon nonparametric test; t=1.96, p=0.05). On the right are the he number of significant voxels in individuals in a region of interest in the hippocampus during Prefrontal Cortex stimulation (100% RMT minus rest over prefrontal cortex) for the LTG day and the placebo day. Compared with placebo, LTG significantly increased the number of active voxels in hippocampus (Wilcoxon nonparametric test; t=1.99, p=0.04).

FIGS. 13A and 13B provide graphs showing average time series for TMS activation on and off LTG. These are cycle-averaged percent change in BOLD signal from baseline over time-within-cycle curves averaged over all 10 subjects from a voxel cluster in the left primary motor cortex directly beneath the TMS coil, during the motor cortex stimulation run. LTG diffusely inhibits the motor cortex TMS-induced activation percent change in BOLD (Three-way ANOVA results showed that % BOLD signal change of LTG significantly decreased compared with placebo (F=11.89, p=0.007), and % BOLD signal change of 120% RMT significantly increased compared with 100% RMT (F=6.27, p=0.034).). On the right are similar time-series from the prefrontal interleaved TMS/phMRI run, except these are averaged over 8 subjects from a voxel cluster in the left hippocampus. LTG increased the TMS-induced percent change in BOLD in this hippocampal region, (Three-way ANOVA results failed to show any difference in % BOLD signal change between either LTG and placebo (F=1.12 p=0.326) or 100% RMT stimulation and 120% RMT stimulation (F=0.32, p=0.591). However, LTM increased % BOLD change at time point 14-17 compared with placebo (n=8×4, t=−2.69, p=0.009)).

### DETAILED DESCRIPTION

In one aspect, the present invention relates to a method of using TMS interleaved with functional brain mapping to test and measure cortical sensitivity and brain communication. In one embodiment, functional brain mapping using fMRI is used in conjunction with specific methods of placing the TMS device over the identified region(s) of the brain, and paired pulse TMS is applied to measure or test sensitivity of the identified region(s).

Embodiments of the present invention, however, are designed to extend beyond these specific technical methods, and cover as well any method of functional brain imaging (including but not limited to PET, SPECT), as well as any method for positioning the TMS device, within or outside of the actual scanner. Moreover, the ability of TMS to produce focal lesions is not specific to any one form of TMS device (figure eight, round, etc), or any one TMS manufacturer.

The capability to perform PP-TMS/fMRI provides a powerful new methodology for noninvasive in-vivo neurophysiology. The present invention provides tools and methods to transform coordinates of target site chosen in MR volume image of subject's brain to settings on TMS coil holder/positioner required to stimulate over that site in a TMS/fMRI study and, conversely, to transform the settings on the TMS coil holder/positioner into the line of peak magnetic field through the MR image volume.

Though fMRI has far better spatial resolution than EEG, its temporal resolution is relatively low, seconds as opposed to milliseconds. The goal of the Paired-Pulse TMS/fMRI work proposed here is an effort to bridge that gap by making use of the observation that TMS applied after a precisely timed delay can be used to modulate responses.

Beckers and Zeki (16) showed that the stimulation of primary visual cortex impairs visual acuity only when delivered at a latency of 60-90 msec following stimulus delivery. In rats, Ogawa et al (17) looked at the fMRI signal as a function of the interval between paired electrical stimulations of the rat forepaws and found a significant suppression of the BOLD signal when the interpulse interval was between 30-40 ms. Chen et al (18) performed a study in human volunteers, in which they plotted the level of BOLD response as a function of the interval between short visual stimuli and found a dip in that response at about 300 ms. It is clear that by using fMRI to observe the time dependence of TMS interference with previous stimulations or cognitive tasks, it is possible to investigate brain communications at time resolutions far greater than that of the hemodynamic response, and approaching that of EEG but with far greater spatial resolution (19, 20).

In general, this technique will make a major contribution to brain research, opening up a whole new area of noninvasive in-vivo research into brain cortex excitability and connectivity and providing an objective means for applying and measuring the efficacy of therapeutic intervention. For example, there is indirect evidence that intracortical inhibition and facilitation (21-24) are caused by separate mechanisms, as opposed to intracortical facilitation being a rebound of the preceding inhibition. However, this evidence has all been acquired through MEPs measured remotely at the target muscle group. With this new PP-TMS/fMRI technique, we will be able to 1) position the coil accurately and repeatable relative to brain anatomy, 2) measure the exact magnetic field distribution of the TMS coil stimulation relative to the brain cortex (25), and 3) observe the local response with millimeter resolution (20). This will provide a significant step forward in the ability to do noninvasive in-vivo neurophysiology.

In some embodiments, real-time blood oxygen level dependent (BOLD) functional MRI (fMRI) analysis offers one approach to functional brain imaging. This approach enables the rapid interpretation of functional imaging results, even while the subject is still in the scanner performing the task. This method is very useful in the pre-surgical mapping of language areas within the brain. In one such embodiment, the subject is next placed in a fMRI scanner such as 1.5 Tesla Philips or Picker Edge 1.5 T scanner and a structural picture of the brain is acquired.

Though the TMS stimulators to be used may not differ from the standard product, the multiplexing unit required to channel the bi-phasic output of these two units through a single TMS coil is an improvement that can be custom built. This improvement will increase both the signal-to-noise and improve the timing control of the interleaving of TMS with fMRI.

A novel holder/positioner greatly increasing the accuracy of coil positioning and allowing the TMS coils position to be referenced to brain anatomy via MR images can be used in some embodiments. In one preferred embodiment, a positioning system is used such as described in copending, commonly assigned U.S. Provisional Application No. 60/381,411 (Bohning et al.), filed May 17, 2002 entitled “A TMS Coil Positioner System” and PCT/US03/15300. These application are hereby incorporated by reference herein for all purposes.

Other embodiments can incorporate other positioning technology. The paired-pulse multiplexer and control circuitry may be integrated with the TMS/fMRI hardware.

There are two main parts to the software development enhancing existing TMS/fMRI software to perform Paired-Pulse TMS/fMRI. The first is a module, which will send the appropriate signals to the multiplexer control circuitry to create a pair of TMS pulses (S1 and S2) with any desired amplitudes (A1 and A2) and interstimulus interval (ISI). The second is the integration of the paired-pulse module into the software used to interleave TMS with fMRI. In general, this is the additional parameterization needed to specify the paired-pulse, and a generalization of the capabilities of the software for handling cyclic and randomized averaged single trial (AST) fMRI experiments. It is recommended that the hardware and software according to exemplary embodiments be tested for timing accuracy and fail-safe prevention of TMS pulse overlap prior to actual use.

Two studies on healthy volunteers will demonstrate the potential of this noninvasive “paired-pulse” TMS/fMRI technique. In the first study, the modulation in BOLD fMRI response associated with thumb movement induced by a series of paired TMS pulses as a function of the interval between the pulses (ISI) will be used to show that the modulation of the BOLD response is sensitive to variations in ISI of the order of milliseconds. In the second study, the relative response versus ISI of “primary” and “secondary” sites activated by TMS applied over prefrontal cortex will be measured as a means of determining the functional dependence of the two sites.

Under the control of an independent computer, the multiplexing unit channels the pulsed output of two bi-polar TMS stimulators through a single coil in switched alternation so as to create a series of paired TMS pulses with a precisely controlled variable interpulse interval (IPI). In addition, the TMS pulse multiplexing circuitry may have a very low inductance to handle the very brief (≈250 μs) and very high currents (10,000 A) used to generate the TMS pulses and to protect each stimulator from the pulses generated by the other, since they will both be firing through a single coil.

The multiplexer circuitry may include blocking sub-circuitry both in the control lines (signal control) from the computer and in the output of the stimulators (pulse control and multiplexing) to eliminate the possibility of simultaneously firing both stimulators and overlapping the TMS pulses. The S1 and S2 lines from the computer will be fed into the signal control circuit. When a pulse comes down either of the control lines (S1 or S2), the other line will be effectively cut for 1 ms to prevent a spurious computer pulse or noise from triggering the other stimulator for 1 ms. The S1 and S2 outputs from this circuit will then be sent to the Trigger Input Ports of the two Magstim units. (Note: The Trigger Input Port accepts TTL compatible signals via its BNC connector; input polarity and whether leading or trailing edge triggered are switch selected.)

Similarly, in the pulse control and multiplexing circuitry, a second protection circuit can, in some embodiments, be combined with the TMS pulse multiplexing circuitry to make it impossible for two TMS pulses to be combined and accidentally raising the stimulation level even if the stimulators should fire without control signals. This downstream blocking control can be initiated by the synchronization pulses available from the TRIGGER OUTPUT port of the Magstim. (Note: These are TTL level pulses which are, typically, used to drive external recording equipment. Polarity and pulse duration, either 50 μs or 50 ms, are switch selectable.)

Previous work (15, 26, 27) has shown that imaging can be performed without significant problems from RF interference if the TMS stimulator is kept outside of MR scanner's RF shielded room, the TMS coil cable is brought into the rear of the MR magnet through a custom-built RF filter box (Lindgren, Inc) grounded to the RF room, and the stimulation/response signal cables are brought into the RF room with the appropriate filters (28). Ancillary control cables from the MR scanner console and experimental control Macintosh are routed through the ceiling over the RF room to the MR scanner electronics room and the appropriate cabinets and TMS stimulator.

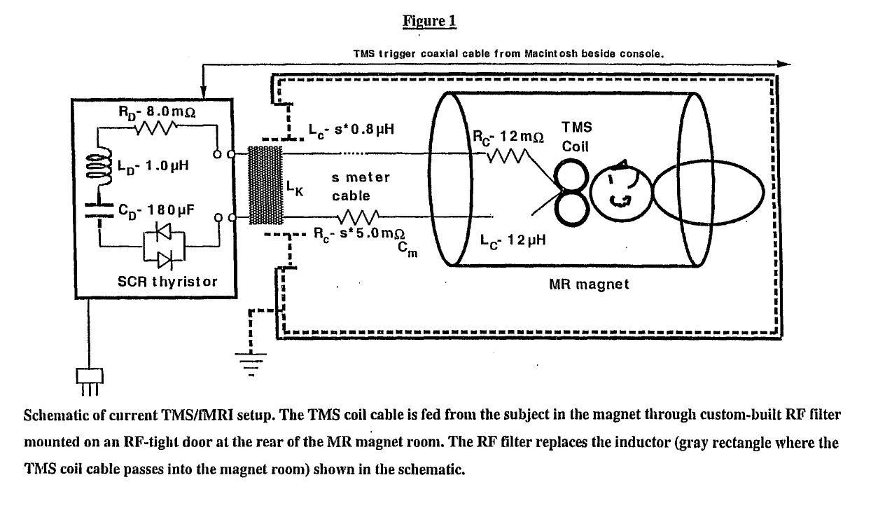

FIG. 1 shows a schematic of an exemplary TMS/fMRI setup, which forms the basis for one embodiment of the present invention. This setup consistently gives a SNR of about 105, indistinguishable from our fMRI scans without TMS.

The timing control for defining protocols and for interleaving the TMS and fMRI image acquisition has also been improved. A G4 Macintosh is used in one preferred embodiment along with the required Input/Output boards. Timing accuracy has been improved from 11.4±3.4 ms to −0.2±0.3 ms.

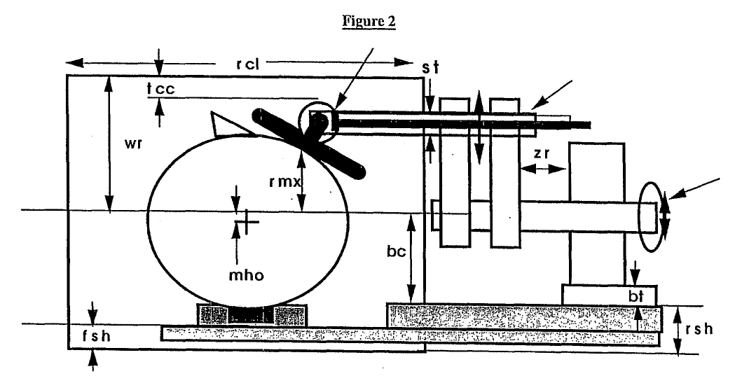

PP-TMS/fMRI uses positioning technology for accurately positioning the TMS coil over a selected area of cerebral cortex. The TMS coil mounting system provides flexible coverage of the scalp to stimulate over any desired area of cerebral cortex yet hold the coil firmly in position during the experiment. This also allows repeatedly positioning the subject with respect to the TMS coil holder and of relating the coil's position to the anatomy of the brain. Schematic drawings of systems to accomplish these goals are seen in FIGS. 2 and/or 3.

Designed with six degrees of freedom, this holder can be used to position the TMS coil over a selected point on the cerebral cortex and then orient the coil so that the plane of the coil is tangent to the skull at that point. In one embodiment, the holder's movements are orthogonal to each other to simplify both the positioning and the computation of the coil's position relative to the isocenter of the MR magnet. Personal computer software allows transformation between coil settings and MR image volumes acquired on the MR scanner while the subject is in position for the PP-TMS/fMRI study.

Coordinates obtained from anatomical locations within the brain on MR images are translated into coil holder settings for accurate and repeatable placement of the TMS coil over those locations. Alternatively, when the coil has been positioned functionally, the settings can be read off and fed into the personal computer software to obtain the coordinates of the coil in the MR scanner's imaging frame of reference.

Though its dimensions and characteristics may be similar to a standard figure-8 TMS coil, e.g., two 70 mm loops and an average inductance of 16.35 pH and maximum field of about 2.2 T, the coil according to exemplary embodiments may be constructed without the normal handle used for handheld applications and have a short stub mounted in the center of the back of the coil for mounting in the holder's radial spar.

The paired-pulse timing control module can be implemented in one embodiment on a Macintosh G4 equipped with a set of input/output (I/O) boards and Labview (National Instruments, Inc.). This module is parameterized and coded in such a way that it can be executed at any desired time in the PP-TMS/fMRI experimental protocol to generate the two stimuli (S1 and S2) with any desired amplitudes (A1 and A2) with any desired interstimulus interval (ISI).

This software can have the same basic structure as that used for the averaged single trial (AST) TMS/fMRI study we did to detect and measure the BOLD signal time course for a single TMS pulse (23), but will be generalized to handle a wider range of fMRI protocols, and the paired-pulse software module can be inserted as an alternative to the single pulse triggering facility.

Numbered citations in the description above correspond to the citations listed in Citation List 1 below.

### CITATION LIST 1

1) Barker A T, Jalinous R, Freeston I L. Non-invasive magnetic stimulation of the human motor cortex. Lancet 1985; 1: 1106-1107.
2) Barker A T, Freeston I L, Jalinous R, Jarratt J A (1987) Magnetic stimulation of the human brain and peripheral nervous system: An introduction and results of an initial clinical evaluation, Neurosurgery 20: 100-109.
3) Day B L, Dressier D, Maertens de Noordhout A, et al (1989) Electric and magnetic stimulation of motor cortex: surface EMO and single motor unit responses. J Physiol (Lond) 412-449-73
4) Jones E G (1981) Anatomy of cerebral cortex: columnar input-output organization. In, The Organization of Cerebral Cortex, Schmitt F O, Worden F G, Adelman G, Dennis S D (Eds), MIT Press, Cambridge, Mass.
5) Brasil-Neto J P, McShane L M, Fuhr P, Hallett M and Cohen L G (1992) Topographic mapping of the human motor cortex with magnetic stimulation: factors affecting accuracy and reproducibility. Electroencephalography and Clinical Neurophysiology 85: 9-16.
6) Wassermann E M, Wang B, Zeffiro T, Sadato N, Pascual-Leone A, Toro C, Hallet M. Locating the motor cortex on the MRI with transcranial magnetic stimulation and PET. Neuroimage 3: 1-9, 1996.
7) Rothwell J C: The use of paired pulse stimulation to investigate the intrinsic circuitry of human motor cortex, in Advances in Magnetic Stimulation: Mathematical Modeling and Clinical Applications. Edited by Nilsson J, Panizza M, Grandori F, Pavia, Italy, PI-ME Press, 1996, pp 99-104.
8) Rossini P M, Barker A T, Berardelli A, et al. Non-invasive electrical and magnetic stimulation of the brain, spinal cord and roots: basic principles and procedures of routine clinical applications: report of an IFCN committee. Electroencephalogr Clin Neurophysiol 91: 79-92, 1994.
9) Ziemann U, Lonnecker S, Steinhoff B J, et al: Effects of antiepileptic drugs on motor cortex excitability in humans: a transcranial magnetic stimulation study. Ann Neurol 40: 367-378, 1996
10) Barker A T, Freeston I L, Jalinous R, et al. Clinical evaluation of conduction time measurements in central motor pathways using magnetic stimulation of human brain (letter). Lancet 1: 1325-1326, 1986.
11) Ziemann U, Basic Neurophysiological Studies with TMS. pp. 45-98, In George M S and Belmaker R H (Eds.) Transcranial Magnetic Stimulation in Neuropsychiatry, American Psychiatric Press, Inc., 2000.
12) Paus T, Jech R, Thompson C J, Comeau R, Peters T, Evans A C: Transcranial Magnetic Stimulation during Positron Tomission Tomography: a new method for studying connectivity of the human cerebral cortex. J. Neurosci. 17(9): 3178-3184, 1997.
13) Fox P, Ingham R, George M S, et al. Imaging human intra-cerebral connectivity by PET during TMS. Neuroreport 1997; 8: 2787-2791.
14) Ilmoniemi R J, Virtanen J, Ruohonen J, et al. Neuronal responses to magnetic stimulation reveal cortical reactivity and connectivity. Neuroreport 1997; 8(16): 3537-3540.
15) Bohning D E, Shastri A, Nahas Z, Lorberbaum J P, Andersen S W, Dannels W R, Haxthausen E-U, Vincent D J and George M S, Echoplanar BOLD fMRI of Brain Activation Induced by Concurrent Transcranial Magnetic Stimulation (TMS), Investigative Radiology 33(6): 336-340, 1998.
16) Beckers G, Zeki S (1995) The consequences of inactivating areas V1 and V5 on visual motion perception. Brain 118: 49-60.
17) Ogawa S, Lee T-M, Stepnoski R, Chen W (2000) Probing neural events by fMRI at the neural time scale of milliseconds. Proc. Intl. Soc. Mag. Reson. Med 8 p. 995.
18) Chen W, Zhu X-H, Ogawa S, Ugurbil K (2000) Probing fast neuronal events and neuronal interactions in human visual cortex during short visual stimulation based on fMRI BOLD response. Proc. Intl. Soc. Mag. Reson. Med 8 p. 501.
19) Krings T, Buchbinder B R, Butler W E et al. (1997) Stereotactic transcranial magnetic stimulation: correlation with direct electrical cortical stimulation. Neurosurgery 41: 1319-1326.
20) Bohning D E, Shastri A, McGavin L, McConnell, Nahas Z, Lorberbaum J P and George M S, Brain Activity for Transcranial Magnetic Stimulation (TMS) Induced and Volitional Movement are Similar in Location and Level. Investigative Radiology, in press.
21) Greenberg B D, Ziemann U, Harmon A, et al: Reduced intracortical inhibition in obsessive-compulsive disorder on transcranial magnetic stimulation. Lancet 352: 881-882, 1998.
22) Rothwell J C, Ferbert A, Caramia M D, Kujirai T, Day B L, Thompson P D, Intracortical inhibitory circuits studied in humans (abstract). Neurology 41(Suppl 1): 192.
23) Kujirai T, Caramia M D, Rothwell J C, Day B L, Thompson P D, Ferbert A, Wroe S, Asselman P, Marsden C D, Corticocortical inhibition in human motor cortex. J Physiol (Land.) 471: 501-519, 1993.
24) Ziemann U. Intracortical inhibition and facilitation in the conventional paired TMS paradigm. Electroencephalography and Clinical Neurophysiology, Suppl 51: 127-136, 1999.
25) Bohning D E, Pecheny A P, Epstein C M, Speer A M, Vincent D J, Dannels W R, and George M S, Mapping Transcranial Magnetic Stimulation (TMS) Fields In-vivo with MRI NeuroReport 8: 2535-2538, (1997).
26) Bohning D E, Shastri A, Blumenthal K A, Nahas Z, Lorberbaum J P, Roberts D R, Teneback C, Vincent D J and George M S, A combined TMS/fMRI study of intensity dependent TMS over motor cortex. Biological Psychiatry 45: 385-394, 1999.
27) Bohning D E, Shastri A, Wassermann E M, Ziemann U, Lorberbaum J P, Nahas Z, Lomarev M P, George M S: BOLD-fMRI Response to Single-Pulse Transcranial Magnetic Stimulation (TMS). JMRI 11 (6): 569-574, 2000.
28) Shastri A, George M S and Bohning D E. Performance of a system for Interleaving Transcranial Magnetic Stimulation with Steady State Functional Magnetic Resonance Imaging. Electroencephalography and Clinical Neurophysiology, Suppl 51: 55-64, 1999.
29) Basser P J, Mattiello J, LeBihan D. MR diffusion tensor spectroscopy and imaging. Biophys. J. 66: 259-267, 1994.
30) Werring D J, Clark C A, Parker G J M, Miller D H, Thompson A I, Barker G J. A direct demonstration of both structure and function in the visual system: Combining diffusion tensor imaging with functional magnetic resonance imaging. Neuroimage. 9(3):352-361, 1999.
31) Jones D K, Simmons A, Williams S C R, Horsfield M A. Non-invasive assessment of axonal fiber connectivity in the human brain via diffusion tensor MRI. Magnetic Resonance in Medicine. 42(1):37-41, 1999.
32) Woods R P, Cherry S R, Mazziota J C. Rapid automated algorithm for aligning and reslicing PET images. J Comput Assist Tomog 16: 620-633.

In the TMS paired-pulse technique, two TMS pulses, separated by a variable interstimulus interval (ISI) are applied to motor cortex while electromyographic (EMG) recordings are made of the motor evoked potentials (MEPs) induced. It is a well characterized physiological tool for testing intracortical inhibition and facilitation, in health and disease, as well as the influence of CNS-active drugs. We have combined the TMS paired-pulse technique with BOLD-fMRI neuroimaging both for testing cortical sensitivity in areas other than motor cortex, and for using the BOLD response amplitude dependence on TMS ISI to investigate brain communication at high time resolution.

For our study, after obtaining informed consent, interleaved paired-pulse TMS/fMRI (1) was performed (to-date) on four healthy volunteers in a whole body 1.5 T MR system (Philips Intera, Rel.8.1.1, Philips Medical Systems, Best, The Netherlands) using a 20 cm diameter circular phased array coil pair and a single shot gradient-echo EPI pulse sequence (TR=1500 ms, TE=40 ms □=80°, matrix 64×64, FOV 256 mm, 11 slices, slice thickness 4 mm, gap 1 mm). A Macintosh G3 laptop with NI DAQCard-AI-16E-4 general purpose I/O board and custom Labview software controlled the firing of two Magstim 220 Stimulators through a BiStim Multiplexer synchronously interleaved with the fMRI acquisition. Using Mathematica, a list of paired-pulse events with ISI of 50, 100, 150, 200, 250, 300, and 1000 ms, pseudo-randomly ordered and spaced, was generated so that the TMS pulses would minimally affect the MR pulse sequence RF pulses. The same event list was later used both to remove TMS compromised images and as the paradigm event list for data analysis with SPM to find areas of BOLD activation.

### Results

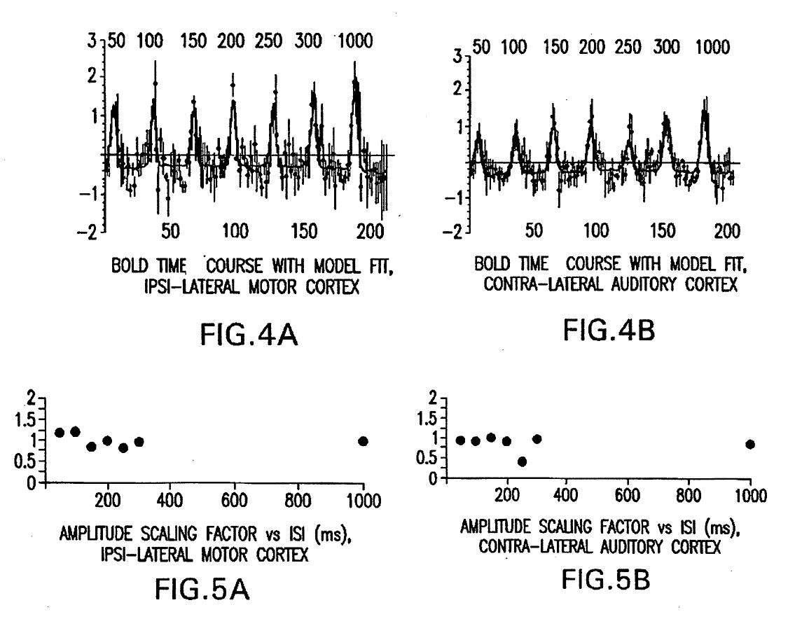

One data set was discarded due to excess movement. Analysis of the other two data sets revealed clusters of pixels with locally high t-values in motor and auditory cortex. Time curves of BOLD response were extracted from the clusters, cycle-averaged and, finally, averaged across the two sets of data. This is shown in FIGS. 4A and 4B.

In FIGS. 4A and 4B, the cycle-averaged paired-pulse data have been rearranged in order of increasing ISI and plotted for ipsi-lateral motor cortex and contra-lateral auditory cortex activations, respectively. A mathematical model made up of a hemodynamice response function multiplied by an exponential recovery function with independent amplitude scaling factors (relative to ISI=1000 amplitude a1000) for the different ISI has been fit to the data and superimposed on the plots as a thick red line.

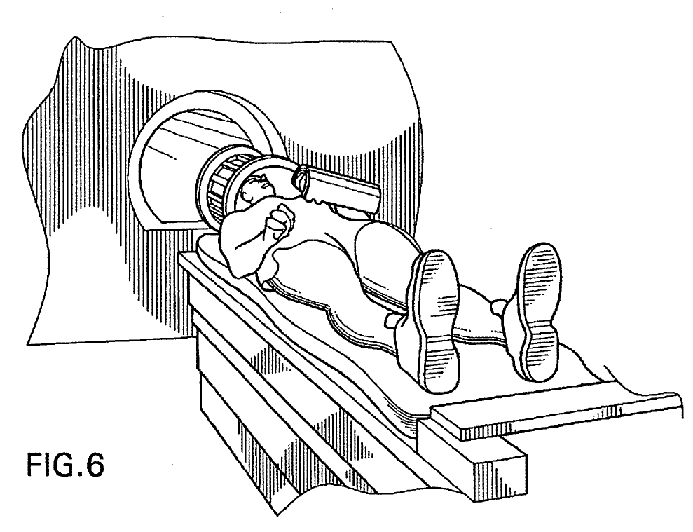

In FIGS. 5A and 5B, the amplitude scaling factors for the fits (a1000=1.0) have been plotted against ISI for motor and auditory cortex activations, respectively. The ipsi-motor data show reduced response near ISI=150 ms, the auditory data show reduced response for an ISI=300 ms.

### Discussion

The data analyzed to date demonstrate the feasibility of combining paired-pulse TMS (2) with fMRI. They also demonstrate that the modulation of the BOLD response amplitude as a function of the ISI between pairs of TMS pulses may be used to test intracortical inhibition and facilitation over the entire brain cortex in health and disease (3), as well as to investigate brain communication at time resolutions an order of magnitude greater than that of the hemodynamic response itself (4, 5). Additional subjects are being recruited for study; their data will be presented as well.

### Acknowledgements

This work was funded in part by a South Carolina Research Initiative Grant.

Citations in the preceding section correspond to those listed in Citation List 2 below.

### CITATION LIST 2

1) Bohning D E, Shastri A, Nahas Z, et al. Investigative Radiology 1998; 33(6): 336-340.
2) Rothwell J C, in Advances in Magnetic Stimulation: Mathematical Modeling and Clinical Applications. Edited by Nilsson J, Panizza M, Grandori F, Pavia, Italy, PI-ME Press, 1996, pp 99-104.
3) Ziemann U, Lonnecker S, Steinhoff B J, et al. Ann Neurol 1996; 40: 367-378.
4) Ogawa S, Lee T-M, Stepnoski R, Chen W Proc. Intl. Soc. Mag. Reson. Med 2000; 8 p. 995.
5) Chen W, Zhu X-H, Ogawa S, Ugurbil K Proc. Intl. Soc. Mag Reson. Med 2000; 8 p. 501.

According to another aspect of the invention, TMS and functional brain mapping may be used to determine efficacy of medications, such as central nervous system (hereinafter “CNS”) active compounds. In one embodiment, functional brain mapping, such as fMRI or BOLD fMRI, is used in conjunction with specific methods of placing the TMS device over the identified regions of the brain. Embodiments of the present invention, however, are designed to extend beyond these specific technical methods, and cover as well any method of functional brain imaging (including but not limited to PET, SPECT, qEEG, MEG), as well as any method for positioning the TMS device, within or outside of the actual scanner. Moreover, the ability of TMS to produce focal lesions is not specific to any one form of TMS device (figure eight, round, etc), or any one TMS manufacturer.

In one embodiment, fMRI is used to determine the brain region or regions that shows activation and/or inhibition while the person is using the CNS-active compound of interest or a particular dosage of such a compound. Once this area is identified using fMRI (or other brain imaging methods), TMS is applied over this region to determine the level of excitation or inhibition relative to excitation or inhibition levels of these areas when the subject is not using the CNS-active compound or is using a differing dosage of such a compound. In some embodiments, measurement of excitation and/or inhibition use paired-pulse TMS as described above.

According to some embodiments, functional brain imaging is applied to a subject to determine brain regions that experience activation and/or inhibition during periods when the subject has taken a CNS-active compound, or a particular dosage thereof.

In some embodiments, the functional brain imaging occurs during both a calibration phase and an analysis phase. In such embodiments, real-time functional brain imaging data is initially gathered during the calibration phase and used during an analysis phase; further real-time data accumulated during the analysis phase can in certain embodiments then be used as feedback to further tune the calibration phase data and enhance the ability to measure efficacy. In yet further embodiments, no calibration phase is required; rather, real-time functional brain imaging data is accumulated during analysis. This imaging data is refined during analysis so that the efficacy measurement improves over the course of analysis. Any suitable functional brain imaging technique can be used including without limitation, including fMRI, PET, SPECT, qEEG and MEG.

In some embodiments, real-time blood oxygen level dependent (BOLD) functional MRI (fMRI) analysis offers one approach to functional brain imaging. This approach enables the rapid interpretation of functional imaging results, even while the subject is still in the scanner performing the task. This method is very useful in the pre-surgical mapping of language areas within the brain. In its current implementations, fMRI appears sensitive enough to detect brain regions impacted by CNS-active compounds and varying dosages thereof.

In one such embodiment, the subject is placed in an fMRI scanner, such as 1.5 Tesla Philips or Picker Edge 1.5 T scanner, and a structural picture of the brain is acquired as depicted in FIG. 5. Next, a series of questions for which the questioner knows the answer are asked in which the person makes either truthful or deceptive answers.

The structural images acquired are transferred to a translational system that allows targeting specific regions in the brain based on MRI or other functional brain scans. In one preferred embodiment, the translational system can be referred to as Brainsight (Rogue Research Inc.). Brainsight is an image analysis and frameless stereotaxy software system that enables the use of landmarks on the face and head (that are also identifiable on the MRI) to localize very specific areas of the brain. Other translational systems can be used within the scope of the present invention.

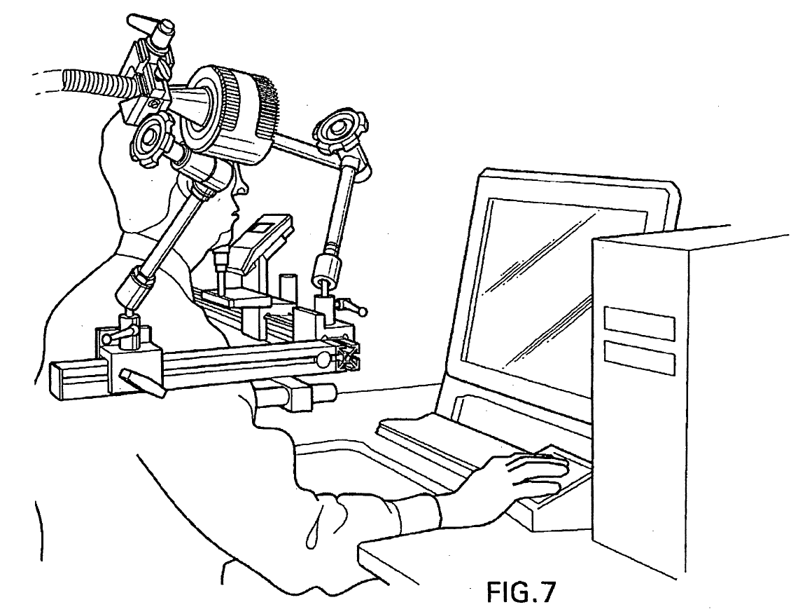

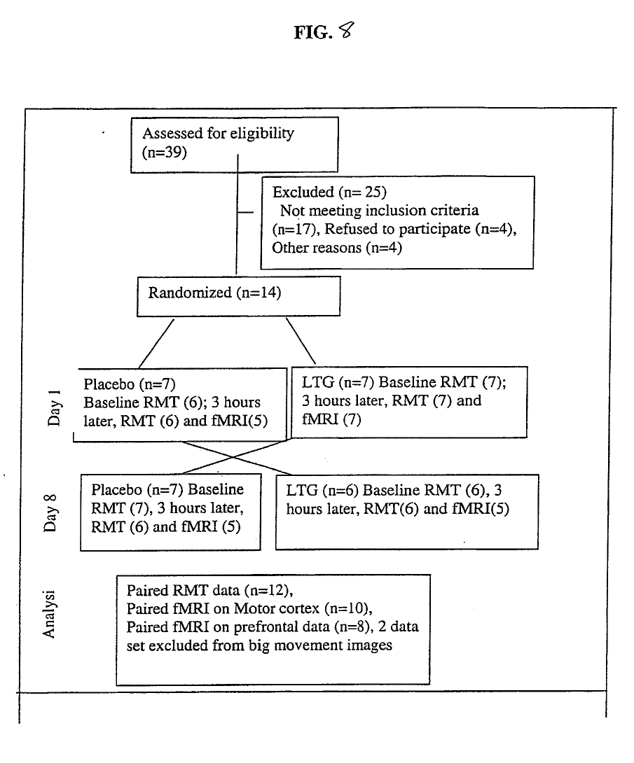

Using the fMRI analysis, the brain regions that show significant activation during deception are identified on the structural brain images. Using Brainsight, the location on the scalp over these brain regions are identified and marked. The distance from skull to cortex over the motor and prefrontal cortex is measured using Brainsight; a particular embodiment of this apparatus is depicted in FIGS. 6 and 7. The TMS motor threshold is determined by using the standard method of the least percent machine output that causes the left thumb to move five out of ten times. The percent output of the TMS machine is adjusted to give 110% of the motor threshold to the prefrontal cortex; this can be accomplished in one preferred embodiment using the Bohning formula discussed below. A variety of translational systems and approaches to TMS delivery useful in the context of the present invention are discussed in copending, commonly assigned U.S. Provisional Application No. 60/367,520 (George et al.), filed Mar. 25, 2002 entitled “Methods and System of Using Transcranial Magnetic Stimulation to Enhance Cognitive Performance” and PCT Application No. PCT/US03/09463. The content of these applications is hereby incorporated by reference herein for all purposes.

The TMS coil is positioned directly over the brain region identified as being activated during deception. Various coil positioning technology can be used. In one preferred embodiment, a positioning system is used such as described in the copending applications mentioned above in the previous section.

### Exemplary Application to Lamotrigine

Lamotrigine (LTG) is a use-dependent sodium channel inhibitor with broad-spectrum anti-convulsant efficacy against a range of epilepsy syndromes¹ (superscript notations throughout this section refer to Citation List 3 below). Recently, several double-blind, placebo-controlled trials have demonstrated the acute and prophylactic antidepressant activity of LTG in bipolar disorder²⁻⁴. Anticonvulsant mood stabilizers may work through the same mechanisms needed for seizure control, but in different brain regions. Thus, some have suggested that LTG stabilizes mood by reducing cortical excitability in areas relevant to the pathogenesis of mood disorder⁵.

As described above, transcranial magnetic stimulation (TMS) is a non-invasive means to stimulate the cerebral cortex, as well as to assess motor cortex excitability^{6, 7}. TMS has been used to examine the pharmacologic effects of anticonvulsant drugs on the excitability of motor corticospinal pathways in both patients with epilepsy and normal subjects^{7,8}. In volunteers or patients with complex partial seizures, LTG significantly increased the resting motor threshold (RMT)^{6,8,9}. Thus, TMS combined with Motor Evoked Potential (MEP)'s can provide useful information about medication effects, but the information is limited to drug effects on motor circuits. TMS over all non-motor brain areas does not produce an easily observable behavioral response, so TMS alone cannot provide information about medication effects in these other important brain regions.

Combining TMS with non-invasive imaging techniques allows one to observe TMS effects throughout the brain. Initial studies used fluorodeoxyglucose (FDG)^{10,11} or oxygen (O15)^{12,13} positron emission tomography (PET). Our group at the Medical University of South Carolina (MUSC) pioneered and developed a technique for interleaving TMS with blood oxygen level dependent (BOLD)-functional magnetic resonance imaging (fMRI)^{14,15}. TMS-induced brain activation does not depend on subject attention, skill or effort, which can influence the amount and location of brain activation in other activation tasks¹⁶. Thus interleaved TMS/fMRI is a non-invasive method to stimulate the cortex and connected brain regions reliably and repeatedly¹⁷.

To our knowledge, no one has yet used interleaved TMS/fMRI to examine medication effects (a process we now refer to as interleaved TMS/pharmacological MRI-phMRI).

In the present study we used interleaved TMS/phMRI to image brain activity during TMS over motor cortex and prefrontal cortex in healthy subjects after receiving a single oral dose of placebo or LTG. We sought to compare RMT and the BOLD TMS-induced pattern of brain activation after LTG or placebo. We hypothesized that, compared to placebo, a single oral dose of LTG would inhibit brain excitability. This LTG blunting would be seen in increased RMT and reduced TMS-induced BOLD activation over motor cortex. We further speculated that LTG would blunt TMS-induced brain activation during TMS over the prefrontal cortex as well as in associated limbic regions. This proof-of concept study sought to test specifically whether interleaved TMS/phMRI might prove a useful tool in understanding LTG's mood-stabilizing mechanisms of action. We also sought to understand whether the interleaved technique might be used to investigate, in general, pharmacological compounds.

Based on our study, interleaved transcranial magnetic stimulation/pharmacological MRI suggests that Lamotrigine inhibits cortical and enhances limbic excitability in healthy young men.

All subjects included in this study were given a detailed explanation of the procedure and signed a written informed consent form approved by the MUSC Investigational Research Board (IRB) and the Food and Drug Administration (FDA). Fourteen healthy young men (aged 18-30) were recruited by local advertisement and then had a screening history and physical examination, structured diagnostic interview¹⁸, baseline laboratory work (basic metabolic panel, liver panel, and hematology), and urine drug screen for drugs of abuse. All subjects were right-handed (as determined by the Annett Handedness Questionnaire¹⁹) and were non-smokers.

### Study

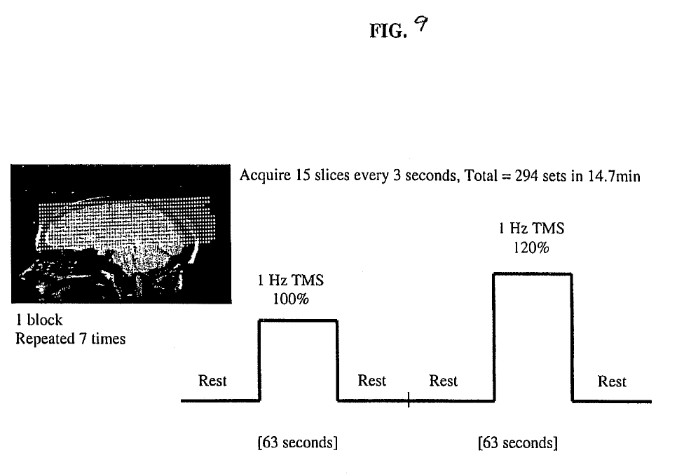

Study design: We performed a randomized, double-blind, crossover trial involving two visits at least one week apart (FIG. 8). The subjects received either a single oral dose of 325 mg of LTG or placebo on the first visit, and then they were given whatever they did not initially receive on the second visit. A single oral dose of 325 mg of LTG has been shown to transiently produce (for 3 hours), serum concentrations equal to steady state levels at clinically relevant chronic doses²⁰. Serum LTG levels, RMTs, and interleaved TMS/phMRI images during both motor and then prefrontal TMS were then gathered for each subject. One week later they received LTG or placebo again, followed by identical RMT and interleaved TMS/phMRI studies.

General Procedure: After arriving at the laboratory in the early afternoon, baseline RMT was determined and baseline plasma levels of LTG were drawn. They were then given a single oral dose of 325 mg LTG or placebo. They then waited quietly for 3 hours. Three hours after taking the oral pill, RMT was determined and serum plasma levels were again drawn.

TMS: Focal TMS was delivered by a MAGSTIM Super Rapid stimulator (Magstim Co, Whitland, Dyfed, U.K) and applied through a focal figure-of-eight magnetic coil (each wing 70 mm in diameter). The optimal position of the magnetic coil for eliciting a MEP in the right abductor pollicis brevis (APB) was determined by holding the coil tangential to the scalp, and moving it in small steps over the presumed area of the left primary motor cortex at a slightly suprathreshold stimulus intensity. The coil was always held horizontally with the handle pointing backward and laterally at 45 degrees from the midline. This position was marked with a pen on a reusable latex swimming cap in order to assure constant placement of the coil throughout the visits. Stimulus intensity and threshold values were expressed as percent of the maximal stimulator output.

Resting Motor Threshold (RMT): Surface electromyographic (EMG) was recorded from the APB using 9-mm Ag—AgCl electrodes in a belly-tendon montage. The placement of electrodes on the thumb and hand was marked with a pen for exact re-placement in consecutive visits on the same day. The raw EMG signal was amplified by a factor of 100 gain and band-pass filtered, 2.0 kHz (low) to 70 kHz (high) with a High Performance Band pass Filter Model V-7548 (LAB Linc. Co). The EMG was recorded on a G3 Macintosh with MacCRO (version 2.1).

RMT was determined in the resting APB in 4 steps: In step one and step three, thresholds were approached from a slightly suprathreshold intensity by reducing the stimulus intensity in 1% steps with a 5 sec interval between pulses, whereas in steps two and four, thresholds were approached from a slightly subthreshold intensity by increasing the stimulus intensity. RMT was defined as the first intensity that produced a MEP of greater than 50 μV in 3 out of 6 trials in the resting target muscle. A mean RMT for baseline or after medication was calculated by averaging the four values. Determination of the RMT using this technique usually lasted 30 minutes.

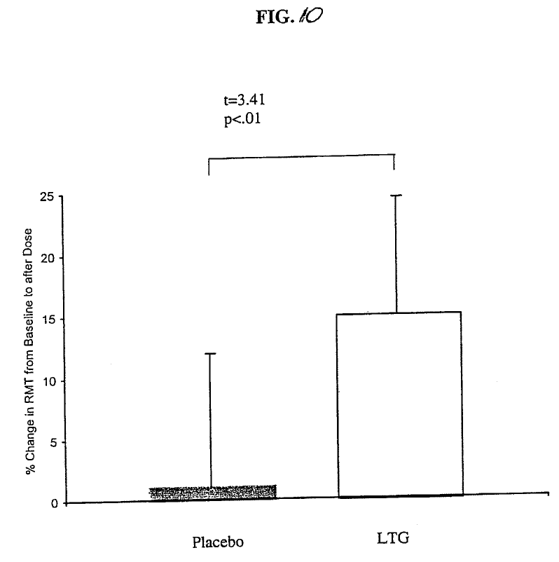

Combined TMS and MRI: Immediately following RMT determination, interleaved TMS/phMRI acquisitions were performed in a Picker EDGE 1.5 T MR scanner with actively shielded magnet and high-performance gradients (27 mT/m, 72 T/m/sec) using a typical gradient echo, echo planar imaging (EPI) fMRI sequence (tip angle=90, TR=3 sec, FOV 27.0 cm, fifteen 7 mm thick slices, 1 mm gap). TMS was delivered using a Dantec MagPro with a special nonferromagnetic TMS coil of figure-8 design with an 8-meter cable (Dantec Medical A/S, Skovlunde, Denmark) and a room set up identical to prior TMS/fMRI studies from our group. TMS pulses and the fMRI sequence were interleaved as described before²¹. Each cycle, illustrated in FIG. 9, consisted of six 21-sec sub-cycles—four Test and two task (100% RMT stimulation and 120% RMT stimulation). During each sub-cycle, the scanner acquired seven sets of 15 transverse images. During the task sub-cycles the TMS was triggered 100 ms after every fifth image acquisition to produce a TMS stimulation rate of 1 Hz. The entire TMS/fMRI sequence lasted 882 sec (14.7 min).

TMS Coil Placement in the MRI scanner: Motor cortex: Before being placed into the MRI scanner, subjects had their resting motor threshold (RMT) quickly determined with the Dantec TMS while sitting on the MRI gantry. For many reasons (different capacitors, coil design, length of cable, MRI filter), the RMT determined with the Magstim in the BSL was not the same RMT needed inside the MRI scanner with the Dantec. After this new MRI RMT was determined, the TMS coil was rigidly mounted in the MR head coil with a specially designed TMS coil-holder, adjustable in six dimensions²². Subjects wore swim caps and special earplugs. With the head coil on the gantry outside the scanner bore, subjects inserted their head into the head coil and adjusted their position while the TMS coil was intermittently pulsed with 100% RMT. Subjects adjusted their head until pulsing the coil caused visible movement of the contralateral (right) hand APB (3 out of 6). As soon as a subject's new MRI RMT-correct scalp location was determined, the holder's six dimensions and earplugs were locked. These head holder settings and RMT were recorded and used with the second visit. During the second MRI visit a week later, the headholder was set with the previous week's coordinates for that subject, and the previous RMT was used for the second visit.

On each visit, immediately after the Motor Cortex MRI study, subjects were removed from the scanner and the TMS device was moved to position it over the left prefrontal cortex. The left prefrontal cortex stimulation site was defined as a location 5-cm rostral and in a parasagittal plane from the site of maximal APB stimulation. Subjects then reentered the scanner for the prefrontal TMS scan, which was identical to the Motor study described above except for the TMS coil location.

### Image Analysis

Individual fMRI Data Analyses: MR scans were transferred into ANALYZE format and then further processed on Sun workstations (Sun Microsystems, Palo Alto, Calif.). Scans were checked using MEDx3.3 (Sensor Systems Inc, Sterling, Va.) for movement across runs, and then were coregistered to a mean image using automatic image registration. For all subjects, movement across the 14.7-minute study was less than 2 mm in all 3 axes. After correction of motion, we used a delayed boxcar model, employed a high-pass filter to remove signal drift, cardiac and respiratory effects, and other low frequency artifacts. Then, we spatially transformed each subject's data into the Talairach Atlas (input voxel dimensions, 2.1×2.1×8 mm, to output voxel dimensions, 4×4×4 mm), smoothed (4×2 mm) the data and generated z map with the Statistical Parametric Mapping (SPM) 96 module in MEDx3.3. We assumed an uncorrected F threshold UF P>0.99 to preserve as many voxels as possible for the cluster analysis. Only clusters showing a statistical weight of P<0.05 were considered to be significantly activated.

Group fMRI Data Analyses: All subject's unthresholded z maps were combined based on comparison of condition (TMS vs Rest), intensity (100% RMT-TMS vs 120% RMT-TMS), visit (LTG vs Placebo). The combined group z maps were thresholded using z≧3.09 (p≦0.001) and cluster statistical weight (spatial extent threshold) of p<0.05. We used either paired or unpaired t-tests in MEDx3.3 for all comparisons of interest and both areas of stimulation.

Magnitude of BOLD time course response: To compare the magnitudes of BOLD signal changes, two types of data were recorded. The different maps of LTG and placebo were used to make a mask of left motor cortex (82 voxels) and a mask of left hippocampus (19 voxels) (FIGS. 11A and 11B bottom panels). The masks used to define location were taken as an index of relative peak intensity above noise. According to the masks' Talairach coordinates, the mean signal intensity of the highest six contiguous voxels (two in each slice) in each subject was extracted from motor cortex or hippocampus with SPM plotting in MEDx. The cycle-mean time courses determined for each subject were transferred to a spreadsheet program, and, by averaging point-by-point within and across subjects, subject-mean and grand-mean time courses were determined (% signal change=100[mean signal at each point−averaged signal in all preceding rests]/averaged signal in all preceding rests).

### Statistical Analysis on Other Variables

The percent change of RMT=100[(post-dose RMT−pre-dose RMT)/pre-dose RMT]. Paired Student's t tests (two tailed) were performed for the percent change of RMT between LTG and placebo. Wilcoxon nonparametric tests were performed for the number of active voxels in the region of interests (ROI) between LTG and placebo. We performed Pearson correlations between the percent change of RMT and the change of active voxel number. Two-way analysis of variance (ANOVA) was performed for % BOLD signal change in the different intensity stimulation and the different medication conditions. All statistical analyses were performed using SPSS 10.0 (Statistical Product and Service Solutions Inc, Chicago Ill.).

### Results

Fourteen subjects were enrolled and were studied. Technical problems with the fMRI scanner or TMS machines meant that not all subjects provided complete data sets. One subject had a baseline RMT greater than the Magstim machine output. After we determined this, the subject was not studied further. Of the 13 subjects studied on two days, 12 subjects (age 25.31±2.70 years) had usable paired TMS RMT data, and of these, two subjects completed the protocol, but their MRI data on at least one of the visits was not usable because of MRI scanner problems. Thus, 10 subjects had complete placebo and LTG interleaved TMS/phMRI data as well as complete RMT data.

### Safety and Tolerability

None of the subjects reported experiencing adverse effects of the drug treatment or the stimulation.

### Resting Motor Threshold

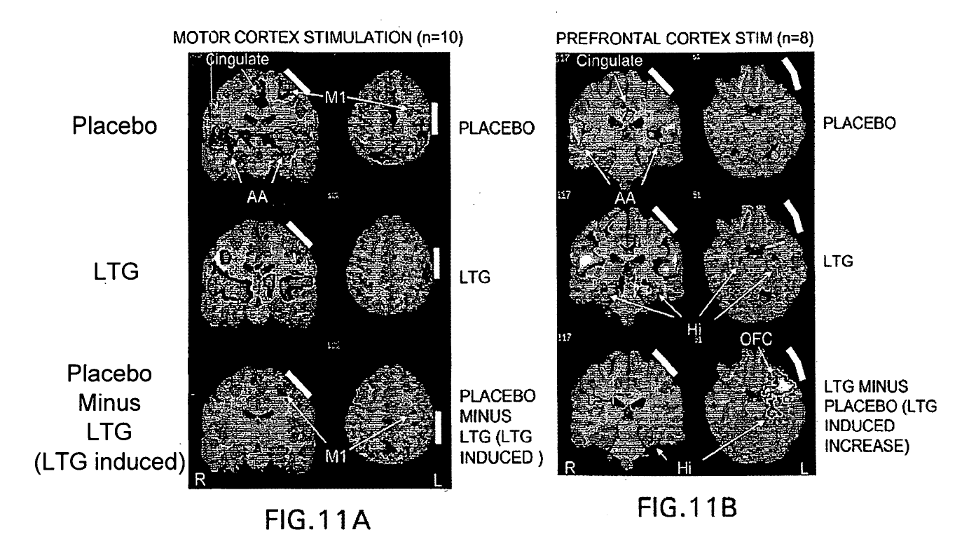

Consistent with our pre-study hypothesis, LTG inhibited the motor cortex and elevated mean motor RMT significantly by 14.9% (SD 9.6) from the same day baseline compared with a placebo increase of 0.6% (SD 10.9) from the same day baseline (Paired Student's t-test, t=3.41, df=11, p<0.01) (see Table 1 and FIG. 10).

TABLE 1
RMT and % change from baseline in 12 subjects
on the two different visits (Placebo, LTG)

| | Placebo | | | LTG | | |
|---|---|---|---|---|---|---|
| | | Post- | % | | Post- | % |
| Subject | Pre | 3 hours | Change | Pre | 3 hours | Change |
| 1 | 67.50 | 67.25 | −.37 | 60.50 | 68.25 | 12.81 |
| 2 | 82.50 | 68.75 | −16.67 | 81.50 | 95.25 | 16.87 |
| 3 | 72.75 | 81.25 | 11.68 | 69.50 | 75.25 | 8.27 |
| 4 | 58.25 | 59.00 | 1.29 | 51.75 | 66.25 | 28.02 |
| 5 | 59.75 | 58.75 | −1.67 | 62.00 | 66.50 | 7.26 |
| 6 | 73.75 | 73.00 | −1.02 | 76.00 | 99.00 | 30.26 |
| 7 | 66.25 | 56.75 | −14.34 | 54.50 | 55.25 | 1.38 |
| 8 | 90.00 | 93.00 | 3.33 | 96.50 | 100.00 | 3.63 |
| 9 | 74.00 | 78.00 | 5.41 | 79.25 | 100.00 | 26.10 |
| 10 | 86.50 | 81.75 | −5.49 | 84.50 | 101.00 | 19.53 |
| 11 | 52.75 | 65.75 | 24.64 | 59.25 | 64.50 | 8.86 |
| 12 | 97.75 | 98.25 | 0.51 | 87.25 | 101.00 | 15.76 |
| Mean ± | 73.47 ± | 73.45 ± | 0.61 ± | 71.89 ± | 82.69 ± | 14.90 ± |
| SD | 13.66 | 13.39 | 10.86 | 14.35 | 18.02* | 9.60** |

Units are percent of machine maximum output (Magstim)
*t = 5.20, p < .01 compared with Pre LTG
**t = 3.41, p < .01 compared with Placebo (% change)

Correlation analyses were performed on the RMT data between visits to assess for the repeatability of the RMT, and the natural variation. The baseline RMT on visit one correlated with the baseline RMT on visit 2, indicating good reliability of the RMT within subjects across visits one week apart (r=0.84, n=12, p<0.01). On both visits, the pre-dose RMT correlated well with the post-dose RMT. On the LTG day, the correlation was shifted, with higher RMT following LTG (placebo visit: r=0.89, n=12, p<0.01; LTG visit: r=0.86, n=12, p<0.01). However, we failed to find a correlation between the serum levels of LTG and post-dose RMT (r=0.34, n=12 p=0.33).

### Interleaved TMS/phMRI Data

### Motor Cortex Stimulation

Motor cortex TMS after either placebo or LTG (within day analysis) at both 100% RMT and 120% RMT resulted in diffuse activation in the brain (see Table 2). 120% RMT stimulation caused more activation than did 100% RMT stimulation in the motor cortex underneath the coil on the placebo day (see Table 2 and FIGS. 11A and 11B).

A formal between-day analysis revealed that, compared to placebo, on the day subjects were taking LTG, they had significantly less TMS-induced activation in the motor cortex (underneath the coil) and other regions. (see FIGS. 11A and 11B bottom panel and Table 3).

TABLE 2
Regions of Activation during TMS Stimulation over
Motor Cortex (Within Day Analyses, n = 10)

| | Talairach | | | | |
|---|---|---|---|---|---|
| | coordinates | | | Z- | |
| Conditions | X | Y | Z | score | Region of activation |

Placebo

| | | | | | |
|---|---|---|---|---|---|
| 100% RMT- | 8 | −44 | 12 | 4.60 | Posterior cingulate (BA 29) |
| Rest | −4 | 28 | 32 | 4.31 | Cingulate gyrus (BA 32) |
| | 48 | −16 | 44 | 3.72 | Right Postcentral |
| | | | | | gyrus (BA 3) |
| | −60 | −28 | 20 | 3.52 | Left postcentral |
| | | | | | gyrus (BA 40) |
| | −40 | 16 | 12 | 3.40 | Left Insula |
| | 40 | 12 | 12 | 3.48 | Right insula (BA 13) |
| | −44 | 12 | −4 | 4.25 | Left inferior frontal |
| | | | | | lobe (BA 47) |
| | 4 | 60 | 8 | 4.20 | Right medial frontal |
| | | | | | gyrus (BA 10) |
| | −24 | −12 | 8 | 4.29 | Left putamen |
| | −48 | 0 | 0 | 4.11 | Left temporal lobe (BA 22) |
| | 64 | −24 | 0 | 3.91 | Right temporal lobe (BA 22) |
| | 36 | −20 | 60 | 4.04 | Right precentral gyrus (BA 4) |
| | −40 | −20 | 60 | 3.47 | Left precentral gyrus (BA 4)* |
| | −40 | −52 | 52 | 3.97 | Left parietal lobe (BA 40) |
| 120% RMT- | 8 | −44 | 12 | 4.60 | Posterior cingulate (BA 29) |
| Rest | −4 | 28 | 32 | 4.31 | Cingulate gyrus (BA 32) |
| | 48 | −16 | 44 | 3.71 | Right Postcentral |
| | | | | | gyrus (BA 3) |
| | −60 | −28 | 20 | 3.51 | Left postcentral |
| | | | | | gyrus (BA 40) |
| | 36 | −20 | 60 | 4.04 | Right precentral gyrus (BA 4) |
| | −38 | −24 | 54 | 3.59 | Left precentral gyrus (BA 4)* |
| | 64 | −24 | 0 | 3.91 | Right superior temporal |
| | | | | | gyrus (BA 22, 21) |
| | −40 | 4 | −20 | 3.81 | Left superior temporal |
| | | | | | gyrus (BA 21) |
| 120% RMT- | −44 | 4 | −4 | 4.60 | Left insula (BA 13) |
| 100% RMT | 44 | −12 | 0 | 4.29 | Right insula (BA 13) |
| | 44 | −56 | 16 | 4.36 | Right superior |
| | | | | | temporal (BA 22) |
| | −36 | 0 | −32 | 4.11 | Left temporal lobe |
| | 40 | −4 | 36 | 4.07 | Right precentral gyrus (BA 6) |
| | −36 | −24 | 56 | 3.81 | Left precentral gyrus (BA 4)* |

LTG

| | | | | | |
|---|---|---|---|---|---|
| 100% RMT- | −44 | 40 | 24 | 4.59 | Left middle frontal |
| | | | | | gyrus (BA 46) |
| Rest | 32 | 8 | 52 | 4.06 | Right middle frontal |
| | | | | | gyrus (BA 6) |
| | −4 | 32 | 28 | 3.97 | Left cingulate gyrus (BA 32) |
| | 40 | −4 | 32 | 4.31 | Right precentral gyrus |
| | 36 | 8 | 12 | 4.26 | Right insula (BA 13) |
| | −36 | 20 | 12 | 3.98 | Left insula (BA 13) |
| | 52 | −16 | 40 | 3.18 | Right postcentral |
| | | | | | gyrus (BA 3) |
| 120% RMT- | −60 | 8 | 24 | 3.14 | Left superior temporal gyrus |
| Rest | 40 | −12 | 4 | 3.14 | Right superior temporal |
| | | | | | gyrus (BA 38) |
| | −32 | 8 | −24 | 6.46 | Left thalamus |
| | −36 | 0 | 4 | 4.60 | Left postcentral gyrus (BA 2) |
| | 4 | 28 | −16 | 4.33 | Right cingulate gyrus |
| | 48 | 36 | 12 | 4.62 | Right middle frontal |
| | | | | | gyrus (BA 9) |
| | −4 | 60 | 0 | 4.06 | Left medial frontal |
| | | | | | gyrus (BA 6) |
| 120% RMT- | | | | | No activity |
| 100% | | | | | |

TABLE3
Talairach Coordinates of Significant Regions
on the Effect of LTG (Between Day nalyses)

| | Talairach coordinates | | | | | | |
|---|---|---|---|---|---|---|---|
| Brain regions | X | Y | Z | Hemisphere | Z-score | p < | Voxels |

Motor Cortex Stimulation
(Placebo-LTG)

| | | | | | | | |
|---|---|---|---|---|---|---|---|
| Left Precentral gyrus* | −32 | −24 | 52 | Left | 3.87 | .001 | 82 |
| Posterior Cingulate | −1 | −25 | 50 | Left | 3.95 | .001 | 93 |
| Precuneus | −1 | −62 | 50 | Left | 3.48 | .001 | 132 |
| Cerebellum | 13 | −46 | −23 | Right | 3.34 | .001 | 70 |

(LTG-Placebo) No Significant Activation
Prefrontal Cortex Stimulation
(Placebo-LTG) No Significant Activation
(LTG-Placebo)

| | | | | | | | |
|---|---|---|---|---|---|---|---|
| Temporal lobe | −43 | 15 | −25 | Left | 3.78 | .001 | 112 |
| Hippocampus | −25 | −11 | −25 | Left | 2.26 | .05 | 19 |
| Insula | 39 | 13 | 1 | Right | 2.83 | .01 | 57 |
| Gyrus frontal | 30 | 25 | 41 | Right | 2.83 | .01 | 35 |
| medius | | | | | | | |
| Postcentral gyrus | 53 | −32 | 40 | Right | 2.83 | .01 | 59 |

*underneath TMS coil

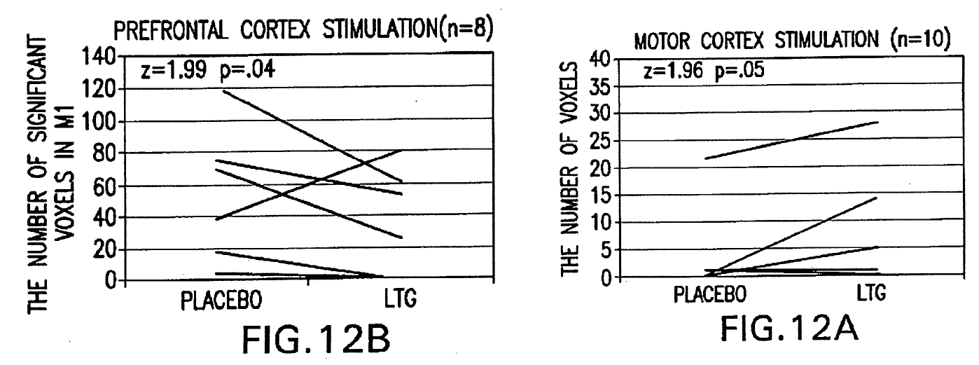

The number of active voxels (120% RMT stimulation minus rest over motor cortex) for placebo and LTG in 10 subjects is shown in FIGS. 12A and 12B. LTG significantly decreased the number of active voxels activated by TMS in the motor cortex. In order to test whether the brain imaging results corresponded with the electrophysiological measures, Pearson correlations were performed on the relationship between the RMT before and after administration of LTG, and the number of active voxels underneath the coil between LTG and placebo days. A significant correlation was found between the increased RMT (see table 1, within LTG day) and inhibited activation in motor cortex (see FIGS. 12A and 12B) (n=10, r=0.81, p<0.01).

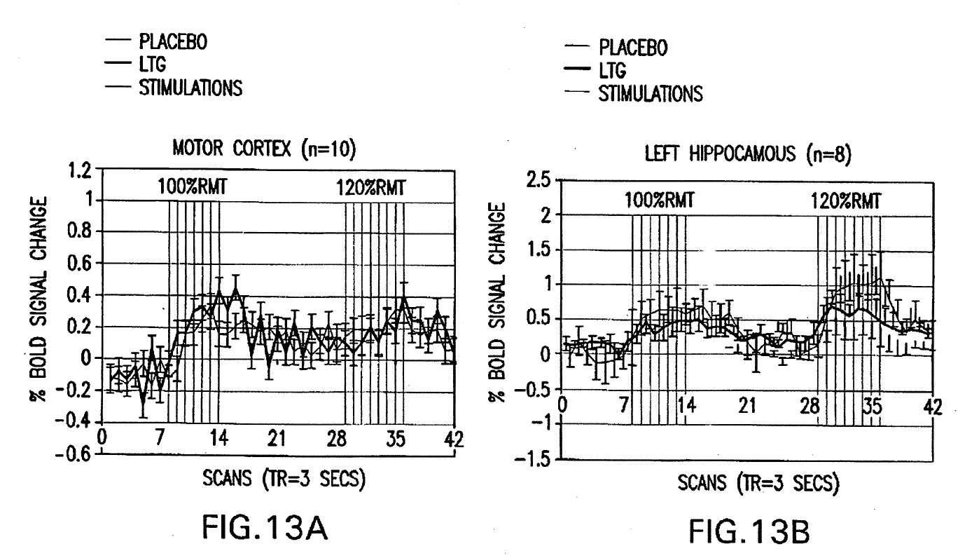

As a further check on the whole brain imaging analysis described above, we examined the timecourse of activation of voxel clusters in the motor cortex directly underneath the coil. For this region, the cycle-averaged time-activity curve was plotted and an estimate obtained of the level of activity in the 120% RMT TMS sub-cycle relative to the preceding rest sub-cycle. FIGS. 13A and 13B summarize the time-activity data pooled across 10 subjects for the motor cortex stimulation. LTG dampened the TMS-induced BOLD response by approximately 50%. Two-way ANOVA results showed that the % BOLD signal change of LTG was significantly decreased compared with placebo (F_{1,20}=11.89, p=0.007), and the % BOLD signal change of 120% RMT was significantly increased compared with 100% RMT (F_{1,6}=6.27, p=0.034). FIGS. 13A and 13B also suggest that LTG's effect is more pronounced towards the end of the stimulation time series than at the beginning.

### Prefrontal Cortex Stimulation

Eight subjects provided usable data from the prefrontal interleaved TMS/phMRI visits. Two subjects whose results were included in the motor cortex analysis were not able to be used in the prefrontal analysis because their prefrontal TMS scans showed more than 2 mm of movement.

Prefrontal cortex stimulation compared to rest, after either placebo or LTG at both 100% RMT and 120% stimulation, induced activation in diffuse brain regions. On either day, unlike with the motor cortex stimulation, there were no statistically significant differences in the pattern of activation between 100% RMT and 120% RMT. Of particular note, brain activity was not significantly increased from rest at the site of stimulation immediately underneath the coil with either 100% RMT or 120% RMT stimulation. (see Table 4 and FIGS. 11A and 11B).

TABLE4
Regions of Activation during TMS Stimulation
over Prefrontal Cortex (Within Day Analyses, n = 8)

| | Talairach | | | | |
|---|---|---|---|---|---|
| | coordinates | | | Z- | |
| Conditions | X | Y | Z | score | Region of activation |

Placebo

| | | | | | |
|---|---|---|---|---|---|
| 100% RMT- | 8 | −44 | 22 | 3.80 | Posterior Cingulate |
| Rest | −4 | 8 | 24 | 3.41 | Anterior Cingulate gyrus |
| | 24 | −32 | 64 | 3.72 | Right Postcentral |
| | | | | | gyrus (BA 3) |
| | −60 | −28 | 20 | 3.52 | Left postcentral |
| | | | | | gyrus (BA 40) |
| | −44 | 8 | 4 | 3.40 | Left Insula (BA 13) |
| | 28 | 40 | 36 | 4.52 | Right medial frontal |
| | | | | | gyrus (BA 10) |
| | −28 | −48 | 16 | 4.28 | Left cerebellum |
| | −48 | 16 | 8 | 5.60 | Left temporal lobe (BA 22) |
| | 56 | −56 | 20 | 3.91 | Right superior temporal lobe |
| | −60 | −4 | 12 | 4.33 | Left precentral gyrus |
| 120% RMT- | 16 | 28 | 20 | 3.21 | Anterior Cingulate gyrus |
| Rest | 56 | −24 | 16 | 3.91 | Right Postcentral |
| | | | | | gyrus (BA 40) |
| | 60 | 0 | 12 | 4.04 | Right precentral gyrus (BA 6) |
| | −64 | 0 | 20 | 3.59 | Left precentral gyrus (BA 6) |
| | 44 | 16 | −20 | 3.91 | Right superior temporal |
| | | | | | gyrus (BA 38) |
| | −36 | −36 | 8 | 4.60 | Left superior temporal gyrus |
| 120% RMT- | | | | | No activity |
| 100% MT | | | | | |

LTG

| | | | | | |
|---|---|---|---|---|---|
| 100% RMT- | −32 | 4 | 60 | 4.04 | Left middle frontal |
| Rest | | | | | gyrus (BA 6) |
| | 40 | 48 | 16 | 3.96 | Right middle frontal gyrus |
| | −8 | −8 | 28 | 4.08 | Left cingulated gyrus |
| | 36 | −4 | 28 | 4.02 | Right precentral gyrus |
| | 52 | −28 | 20 | 4.26 | Right insula (BA 13) |
| | 52 | −16 | 40 | 3.18 | Right postcentral |
| | | | | | gyrus (BA 3) |
| | 20 | −8 | −16 | 3.66 | Right hippocampus, |
| | | | | | Amygdala |
| | −24 | −8 | −24 | 3.53 | Left Hippocampus |
| 120% RMT- | −60 | 4 | −4 | 4.14 | Left superior temporal gyrus |
| Rest | 56 | −12 | 8 | 4.53 | Right superior temporal gyrus |
| | −56 | −24 | 36 | 4.12 | Left postcentral gyrus (BA 2) |
| | −8 | 4 | 36 | 3.98 | Left cingulate gyrus |
| | −48 | 44 | −4 | 4.15 | Left medial frontal |
| | | | | | gyrus (BA 6) |
| | 52 | −8 | 44 | 4.31 | Right precentral gyrus |
| 120% RMT- | | | | | No activity |
| 100% | | | | | |

A formal between-day analysis showed that, with respect to the rest condition, there was increased brain activity in the hippocampus and the orbital frontal gyrus during 100% RMT stimulation when in the presence of LTG compared to placebo. (FIGS. 11A and 11B bottom panel, Table 3).

The number of active voxels (100% RMT stimulation minus rest over prefrontal cortex) after placebo or LTG in 8 subjects are shown in FIGS. 12A and 12B. There were significantly more TMS-induced active voxels in the left hippocampus after LTG than after placebo.

We examined the timecourse of activation of the cluster of voxels in the left hippocampus. FIGS. 13A and 13B summarize the time-activity data pooled across 8 subjects. Two-way ANOVA results of the entire time series failed to show significant differences in % BOLD signal change between either LTG and placebo (F_{1,20}=1.12 p=0.326) or 100% RMT stimulation and 120% RMT stimulation (F_{1,6}=0.32, p=0.591). However, a formal comparison of activity during the TMS phase (time points 14-17) revealed that LTG significantly increased % BOLD change compared with placebo (t=−2.69, df=15, p=0.009).

### Interleaved TMS/phMRI

To our knowledge, this is the first report to use the interleaved TMS/fMRI technique to investigate the regional brain effects of a central nervous system (CNS)-active compound (referred to as interleaved TMS/phMRI). We found, consistent with our hypothesis, that LTG inhibited the motor cortex when we applied TMS over the motor area for thumb movement. This LTG inhibition was evident both in the electrophysiological measurements, and the regional brain activity. Over the motor cortex, the brain imaging and electrophysiological domains as well were highly correlated. Surprisingly, we found that LTG had a different effect when we applied TMS over the prefrontal cortex. Not only did LTG not inhibit the BOLD response, it actually increased activity in the limbic system.

The results demonstrate that it is possible to combine TMS and phMRI to evaluate both decreasing and increasing regional brain effects of CNS compounds. We thus conclude that interleaved TMS/phMRI is feasible as a new neuroscience tool, and may have several important uses.

Analysis of the group fMRI data of TMS over motor cortex on the placebo day revealed robust EMS-induced activation of the ipsilateral motor cortex^{14,23} as well as bilateral activation of the auditory cortex. Interestedly, the present data also showed that TMS caused activation of the contralateral (right) motor cortex as well. Although the control of movement is one of the clearest hemispherically-lateralized functions in the brain²⁴, human functional neuroimaging studies of hand motor control commonly report bilateral activation in primary motor cortex^{25,26}. We also compared BOLD-fMRI responses at two different stimulation intensities, and found that high intensity motor cortex stimulation (120% RMT) was associated with significantly increased activation compared to lower intensity (100% RMT) stimulation¹⁴, on the placebo day only. These results on the placebo medication day replicate our previous studies of motor cortex TMS/fMRI, all of which have shown dose-dependent TMS effects^{15,27} Finally, we also analysed the timecourse of activation in motor cortex and found a 1% BOLD activation relative to baseline could be observed at 120% RMT stimulation.

### Detecting Pharmacological Effects on RMT and the BOLD Response

Several prior TMS studies have shown that LTG increases the threshold of MEPs elicited by TMS^{6,8,28}. In the present study, we confirmed the inhibitory effect of LTG on MEPs. LTG caused a 14.9% increase in RMT in healthy young adults, which agrees with previous TMS studies with the compound^{6,8,29}. A region of interest analysis of the fMRI data showed that LTG reduced activation in the motor cortex, directly under the coil, and in other diffuse areas of the cortex. As one might predict, the increase in MEP threshold correlated with the decrease in BOLD-fMRI measures in the presence of LTG.

### Detecting a BOLD Response to TMS Over the Prefrontal Cortex

In addition to TMS over the motor cortex, we then applied the interleaved TMS/phMRI technique over the prefrontal cortex, using a probabilistic positioning method. In this case we were limited to examining the fMRI measurements alone, since there is no overt behavioral response, like an MEP, to prefrontal cortical stimulation. We have shown previously that unilateral TMS applied over the prefrontal cortex (left) has bilateral effects, and that higher intensity stimulation produces greater ipsi- and contralateral activation³⁰. In addition, other PET and SPECT studies have shown that increases and decreases in blood flow or metabolism occur during and shortly after repetitive TMS (rTMS) applied over the prefrontal cortex^{11;31;32}. The present study confirmed the bilateral cortical effects of TMS when applied to the prefrontal cortex. However, on the placebo day, we found no significant difference between the activation at 100% RMT and 120% RMT with prefrontal TMS. Although we failed to find prefrontal cortex induced activation underneath the prefrontal coil, the results showed significant bilateral activation of the limbic system only when the subjects were taking LTG. These paradoxical prefrontal results, where LTG is not inhibiting but rather increasing limbic activation, softly suggest that LTG may have a unique relationship with limbic activation, that differs from its effects in the motor circuit. This may be due to differential regional effects of LTG, or due to some interaction of cortical-limbic loops and relative governance. Although these are intriguing results, they are highly speculative given the non-hypothesized nature of these findings and the small sample size. An additional study attempting replication is recommended.

### What is LTG Doing to the Interleaved TMS/Bold Response?

LTG's anticonvulsant activity has generally been attributed to its ability to stabilize the inactive form of brain sodium channels^{33,34}, though this alone may not account for its broad efficacy³⁵. Indeed, LTG has also been shown to have activity at other ion channels³⁶⁻³⁹. Although the molecular target or targets through which LTG exerts its therapeutic effect may not be known precisely, evidence suggests that reduction in glutamate release and enhancement of GABA release may be important downstream effects⁴⁰⁻⁴². Of particular interest is a recent study from Calabresi et al (1999)⁴³, which found that LTG reduced cortico-striatal excitatory transmission in the rat via a pre-synaptic mechanism that may be independent of sodium channel blockade.

A key finding of the present study was that BOLD responses induced by TMS in the motor cortex could be inhibited by LTG (a BOLD signal decreased of 50% relative to baseline). Furthermore, the effect of LTG was stronger on TMS at 120% RMT than at 100% TMS and the timecourse analysis (FIGS. 11A and 11B) suggests a greater effect of LTG towards the end of the series of stimulations. These observations are consistent with the decreased positive BOLD fMRI signal of LTG during forepaw stimulation in the rodent⁴⁴. Interestingly, our results also showed that LTG induced RMT inhibition significantly correlated with decreased BOLD fMRI activation in motor cortex when subjects took LTG.

What does this Tell Us about LTG's Mechanism of Action in Bipolar Disorder?

Double-blind, placebo-controlled trials have demonstrated the acute and prophylactic antidepressant activity of LTG in bipolar disorder^{5,45,46}. Various hypotheses have been proposed regarding its mechanism of action on mood. One may speculate that the efficacy of LTG in bipolar disorder is related to its anticonvulsant efficacy, and so also to its anticonvulsant mechanisms of action. However, the clinical profile of LTG in bipolar disorder is different from that of either valproate or carbamazepine, and in fact its spectrum of anticonvulsant efficacy is also somewhat different, notably its efficacy versus absence seizures⁴⁷.

The present study in healthy volunteers may not be relevant to drug effects in patients with bipolar disorder, but the surprising limbic activation obtained in the presence of LTG when TMS was applied to the prefrontal cortex is worth considering with the clinical situation in mind. Studies of the neuropathology in familial Major Depressive Disorder have reported changes in morphology and metabolism in selected areas of the limbic system, such as the hippocampus⁴⁸⁻⁵⁰ so orbital frontal lobe^{51,52} and amygdala⁵³. Frodl⁵⁴ reported smaller hippocampal gray matter volumes in patients with a first episode of major depression compared with healthy subjects. Furthermore, recent data suggests⁵⁵ that bipolar disorder is associated with a significant decrease of glutamic acid decarboxylase (GAD) mRNA-positive neurons and of GAD₆₅ mRNA expression in the hippocampus. Regarding pharmacotherapy, several studies have reported increased regional activation (the left prefrontal cortex, thalamus, and medial frontal gyrus⁵⁶⁻⁶⁰) post-treatment in depressed patients. These findings provide soft evidence of limbic system abnormality in bipolar disorder. The present study showed that LTG could induce increased activity in hippocampus in normal subjects compared with placebo. This leads to speculation that the antidepressant effect of LTG could be mediated by increasing activity in hippocampus or other limbic structures.

Like all studies, this initial proof-of-concept study suffers from limitations that bear on the interpretation of the results. The prefrontal cortex data failed to show our earlier finding of activation underneath the coil³⁰. Although the present data over motor cortex showed dose-dependent TMS effects, we failed to find the same dose-dependent TMS effects over prefrontal cortex (FIGS. 13A and 13B). Additionally, our subjects were healthy results, and the findings cannot necessarily be generalized to patients with mood disorders. This study needs replication in healthy adults, as well as an additional study in patients with mood disorders, before firm acceptance.

### CONCLUSIONS

In conclusion, this current study suggests that the interleaved TMS/phMRI technique has utility in understanding the regional brain effects of LTG, and likely other CNS-active compounds. Using the technique, we found as hypothesized that LTG has an inhibitory effect on motor cortical neuronal excitability measured both by RMT and interleaved TMS/phMRI. On the other hand, LTG may have a complex effect on prefrontal TMS, with cortical inhibition and limbic facilitation. It is unclear if these effects may be relevant to the efficacy of LTG in mood disorders. Further studies are warranted with this promising new technique.

Some embodiments can include a precursor step to functional brain imaging and/or application of TMS that involves evaluating the subject for potential risk. If potential risk is greater than a predetermined level with respect to a particular functional brain imaging technique, or particular parameter set associated therewith, and/or TMS configuration, or particular parameter set associated therewith, an alternative technique, configuration and/or parameter set can be used. Such an alternative technique, configuration and/or parameter set can, in certain embodiments, be subject to its own potential risk evaluation with respect to the subject.

Citations in the preceding section correspond to those listed in Citation List 3 below.

### CITATION LIST 3

1. Messenheimer J A. Lamotrigine. Epilepsia. 1995; 36 Suppl 2:S87-S94.
2. Calabrese J R, Suppes T, Bowden C L, Sachs G S, Swann A C, McElroy S L, Kusumakar V, Ascher J A, Earl N L, Greene P L, Monaghan E T. A double-blind, placebo-controlled, prophylaxis study of lamotrigine in rapid-cycling bipolar disorder. Lamictal 614 Study Group. J Clin Psychiatry. 2000; 61:841-850.
3. Frye M A, Ketter T A, Kimbrell T A, Dunn R T, Speer A M, Osuch E A, Luckenbaugh DA, Cora-Ocatelli G, Leverich G S, Post R M. A placebo-controlled study of lamotrigine and gabapentin monotherapy in refractory mood disorders. J Clin Psychopharnacol. 2000; 20:607-614.
4. Calabrese J R. Lamotrigine and the treatment of bipolar disorder. Introduction. Eur Neuropsychopharmacol. 1999; 9 Suppl 4:S107-S108.
5. Ketter T A, Calabrese J R. Stabilization of mood from below versus above baseline in bipolar disorder: a new nomenclature. J Clin Psychiatry. 2002; 63:146-151.
6. Ziemann U, Lonnecker S, Steinhoff B J, Paulus W. Effects of antiepileptic drugs on motor cortex excitability in humans: a transcranial magnetic stimulation study. Ann Neurol. 1996; 40:367-378.
7. George M S, Lisanby S H, Sackeim H A. Transcranial magnetic stimulation: applications in neuropsychiatry. Arch Gen Psychiatry. 1999; 56:300-311.
8. Manganotti P, Bongiovanni L G, Zanette G, Turazzini M, Fiaschi A. Cortical excitability in patients after loading doses of lamotrigine: a study with magnetic brain stimulation. Epilepsia. 1999; 40:316-321.
9. Ziemann U, Hallett M, Cohen LG. Mechanisms of deafferentation-induced plasticity in human motor cortex. J. Neurosci. 1998; 18:7000-7007.
10. George M S, Wassermann E M, Williams W A, Callahan A, Ketter T A, Basser P, Hallett M, Post R M. Daily repetitive transcranial magnetic stimulation (rTMS) improves mood in depression. Neuroreport. 1995; 6:1853-1856.
11. Kimbrell T A, Little J T, Dunn R T, Frye M A, Greenberg B D, Wassermann E M, Repella J D, Danielson A L, Willis M W, Benson B E, Speer A M, Osuch E, George M S, Post R M. Frequency dependence of antidepressant response to left prefrontal repetitive transcranial magnetic stimulation (rTMS) as a function of baseline cerebral glucose metabolism. Biol Psychiatry. 1999; 46:1603-1613.
12. Paus T, Jech R, Thompson C J, Comeau R, Peters T, Evans A C. Transcranial magnetic stimulation during positron emission tomography: a new method for studying connectivity of the human cerebral cortex. J Neurosci. 1997; 17:3178-3184.
13. Paus T, Wolforth M. Transcranial magnetic stimulation during PET: reaching and verifying the target site. Hum Brain Mapp. 1998; 6:399-402.
14. Bohning D E, Shastri A, McGavin L, McConnell K A, Nahas Z, Lorberbaum J P, Roberts D R, George M S. Motor cortex brain activity induced by 1-Hz transcranial magnetic stimulation is similar in location and level to that for volitional movement. Invest Radiol. 2000; 35:676-683.
15. Bohning D E, Shastri A, McConnell K A, Nahas Z, Lorberbaum J P, Roberts D R, Teneback C, Vincent D J, George M S. A combined TMS/fMRI study of intensity-dependent TMS over motor cortex. Biol Psychiatry. 1999; 45:385-394.
16. George M S, Bohning D E. Measuring brain connectivity with functional imaging and transcranial magnetic stimulation (TMS). In: Desimone B, eds. Neuropsychopharmacology, Fifth Generation of Progress. New York, N.Y.: Lipincott, Williams and Wilkins; 2002:393-410.
17. Denslow S, Lomarev M, George M S, Bohning D E. TMS-Induced versus volitionally-induced BOLD response in the motor cortex: high resolution comparison of location, intensity and repeatability. Hum Brain Mapp. (Submitted, 2002/09).
18. First M B, Spitzer R L, Gibbon M, and Williams J B W. Structured clinical interview for DSM-IV Axis I disorders. Washington D.C. American Psychiatric Association. 1995.
19. Annett M. A classification of hand preference by association analysis. Br J Psychol. 1970; 61:303-321.
20. Steinhoff, B. J. and Tergau, F. Report of a study to investigate the effects of lamotrigine on the magnetic motor evoked potential (MMEP) produced by transcranial magnetic stimulation (TMS) in healthy volunteers (Unpublished Work). Clinical Investigator's Brochure Lamictal, Glaxo Wellcome, Inc. Research Triangle Park, NC 1999.
21. Shastri A, George M S, Bohning D E. Performance of a system for interleaving transcranial magnetic stimulation with steady-state magnetic resonance imaging. Electroencephalogr Clin Neurophysiol Suppl. 1999; 51:55-64.
22. Bohning D E, Denslow S Bohning P A Marsh J A Nahas Z George M S. TMS coil positioner/holder and software system for MR image-guided interleaved TMS/fMRI. In: Tenth Scientific Meeting and Exposition of the International Society for Magnetic Resonance in Medicine, May 23, 2002. Honolulu, Hi. Number 1343. Abstract.
23. Baudewig J, Siebner H R, Bestmann S, Tergau F, Tings T, Paulus W, Frahm J. Functional MRI of cortical activations induced by transcranial magnetic stimulation (TMS). Neuroreport. 2001; 12:3543-3548.
24. Dassonville P, Lewis S M, Zhu X H, Ugurbil K, Kim S G, Ashe J. Effects of movement predictability on cortical motor activation. Neurosci Res. 1998; 32:65-74.
25. Hlustik P, Solodkin A, Gullapalli R P, Noll D C, Small S L. Functional lateralization of the human premotor cortex during sequential movements. Brain Cogn. 2002; 49:54-62.
26. Solodkin A, Hlustik P, Noll D C, Small S L. Lateralization of motor circuits and handedness during finger movements. Eur J Neurol. 2001; 8:425-434.
27. Bohning D E, He L, George M S, Epstein C M. Deconvolution of transcranial magnetic stimulation (TMS) maps. J Neural Transm. 2001; 108:35-52.
28. Villetti G, Bregola G, Bassani F, Bergamaschi M, Rondelli I, Pietra C, Simonato M. Preclinical evaluation of CHF3381 as a novel antiepileptic agent. Neuropharmacology. 2001; 40:866-878.
29. Ziemann U, Tergau F, Wischer S, Hildebrandt J, Paulus W. Pharmacological control of facilitatory I-wave interaction in the human motor cortex. A paired transcranial magnetic stimulation study. Electroencephalogr Clin Neurophysiol. 1998; 109:321-330.
30. Nahas Z, Lomarev M, Roberts D R, Shastri A, Lorberbaum J P, Teneback C, McConnell K, Vincent D J, Li X, George M S, Bohning D E. Unilateral left prefrontal transcranial magnetic stimulation (TMS) produces intensity-dependent bilateral effects as measured by interleaved BOLD fMRI. Biol Psychiatry. 2001; 50:712-720.
31. Speer A M, Kimbrell T A, Wassermann E M, Repella D, Willis M W, Herscovitch P, Post R M. Opposite effects of high and low frequency rTMS on regional brain activity in depressed patients. Biol Psychiatry. 2000; 48:1133-1141.
32. Strafella A P, Paus T, Barrett J, Dagher A. Repetitive transcranial magnetic stimulation of the human prefrontal cortex induces dopamine release in the caudate nucleus. J Neurosci. 2001; 21:RC157.
33. Catterall W A. Molecular properties of brain sodium channels: an important target for anticonvulsant drugs. Adv Neurol. 1999; 79:441-456.
34. Xie X, Lancaster B, Peakman T, Garthwaite J. Interaction of the antiepileptic drug lamotrigine with recombinant rat brain type IIA Na+ channels and with native Na+ channels in rat hippocampal neurones. Pflugers Arch. 1995; 430:437-446.
35. Coulter D A. Antiepileptic drug cellular mechanisms of action: where does lamotrigine fit in? J Child Neurol. 1997; 12 Suppl 1:S2-S9.
36. Grunze H, Von Wegerer J, Greene R W, Walden J. Modulation of calcium and potassium currents by lamotrigine. Neuropsychobiology. 1998; 38:131-138.
37. Poolos N P, Migliore M, Johnston D. Pharmacological upregulation of h-channels reduces the excitability of pyramidal neuron dendrites. Nat Neurosci. 2002.
38. Von Wegerer J, Hesslinger B, Berger M, Walden J. A calcium antagonistic effect of the new antiepileptic drug lamotrigine. Eur Neuropsychopharmacol. 1997; 7:77-81.
39. Xie X, Hagan R M. Cellular and molecular actions of lamotrigine: Possible mechanisms of efficacy in bipolar disorder. Neuropsychobiology. 1998; 38:119-130.
40. Cunningham M O, Jones R S. The anticonvulsant, lamotrigine decreases spontaneous glutamate release but increases spontaneous GABA release in the rat entorhinal cortex in vitro. Neuropharmacology. 2000; 39:2139-2146.
41. Lingamaneni R, Hemmings H C J. Effects of anticonvulsants on veratridine- and KCl-evoked glutamate release from rat cortical synaptosomes. Neurosci Lett. 1999; 276:127-130.
42. Waldmeier P C, Martin P, Stocklin K, Portet C, Schmutz M. Effect of carbamazepine, oxcarbazepine and lamotrigine on the increase in extracellular glutamate elicited by veratridine in rat cortex and striatum. Naunyn Schmiedebergs Arch Pharmacol. 1996; 354:164-172.
43. Calabresi P, Centonze D, Marfia G A, Pisani A, Bernardi G. An in vitro electrophysiological study on the effects of phenytoin, lamotrigine and gabapentin on striatal neurons. Br J Pharmacol. 1999; 126:689-696.
44. Kida I, Hyder F, Behar K L. Inhibition of voltage-dependent sodium channels suppresses the functional magnetic resonance imaging response to forepaw somatosensory activation in the rodent. J Cereb Blood Flow Metab. 2001; 21:585-591.
45. Calabrese J R, Bowden C L, Sachs G S, Ascher J A, Monaghan E, Rudd G D. A double-blind placebo-controlled study of lamotrigine monotherapy in outpatients with bipolar I depression. Lamictal 602 Study Group. J Clin Psychiatry. 1999; 60:79-88.
46. Calabrese J R, Bowden C L, McElroy S L, Cookson J, Andersen J, Keck P E J, Rhodes L, Bolden-Watson C, Zhou J, Ascher J A. Spectrum of activity of lamotrigine in treatment-refractory bipolar disorder. Am J Psychiatry. 1999; 156: 1019-1023.
47. Muzina D J, El-Sayegh S, Calabrese J R. Antiepileptic drugs in psychiatry-focus on randomized controlled trial. Epilepsy Res. 2002; 50:195-202.
48. Rusch B D, Abercrombie H C, Oakes T R, Schaefer S M, Davidson R J. Hippocampal morphometry in depressed patients and control subjects: relations to anxiety symptoms. Biol Psychiatry. 2001; 50:960-964.
49. Thibault O, Hadley R, Landfield P W. Elevated postsynaptic [Ca2+]i and L-type calcium channel activity in aged hippocampal neurons: relationship to impaired synaptic plasticity. J Neurosci. 2001; 21:9744-9756.
50. Auer D P, Putz B, Kraft E, Lipinski B, Schill J, Holsboer F. Reduced glutamate in the anterior cingulate cortex in depression: an in vivo proton magnetic resonance spectroscopy study. Biol Psychiatry. 2000; 47:305-313.
51. MacFall J R, Payne M E, Provenzale J E, Krishnan K R. Medial orbital frontal lesions in late-onset depression. Biol Psychiatry. 2001; 49:803-806.
52. Drevets W C. Functional anatomical abnormalities in limbic and prefrontal cortical structures in major depression. Prog Brain Res. 2000; 126:413-431.
53. Sheline Y I, Gado M H, Price J L. Amygdala core nuclei volumes are decreased in recurrent major depression. Neuroreport. 1998; 9:2023-2028.
54. Frodl T, Meisenzahl E M, Zetzsche T, Born C, Groll C, Jager M, Leinsinger G, Bottlender R, Hahn K, Moller H J. Hippocampal changes in patients with a first episode of major depression. Am J. Psychiatry. 2002; 159:1112-1118.
55. Heckers S, Stone D, Walsh J, Shick J, Koul P, Benes F M. Differential hippocampal expression of glutamic acid decarboxylase 65 and 67 messenger RNA in bipolar disorder and schizophrenia. Arch Gen Psychiatry. 2002; 59:521-529.
56. Bench C J, Frackowiak R S, Dolan R J. Changes in regional cerebral blood flow on recovery from depression. Psychol Med. 1995; 25:247-261.
57. Buchsbaum M S, Wu J, Siegel B V, Hackett E, Trenary M, Abel L, Reynolds C. Effect of sertraline on regional metabolic rate in patients with affective disorder. Biol Psychiatry. 1997; 41:15-22.
58. Mayberg H S, Liotti M, Brannan S K, McGinnis S, Mahurin R K, Jerabek P A, Silva J A, Tekell J L, Martin C C, Lancaster J L, Fox P T. Reciprocal limbic-cortical function and negative mood: converging PET findings in depression and normal sadness. Am J. Psychiatry. 1999; 156:675-682.
59. Baxter L R J, Schwartz J M, Phelps M E, Mazziotta J C, Guze B H, Selin C E, Gerner R H, Sumida R M. Reduction of prefrontal cortex glucose metabolism common to three types of depression. Arch Gen Psychiatry. 1989; 46:243-250.
60. Saxena S, Brody A L, Ho M L, Alborzian S, Maidment K M, Zohrabi N, Ho M K, Huang S C, Wu H M, Baxter L R J. Differential cerebral metabolic changes with paroxetine treatment of obsessive-compulsive disorder vs major depression. Arch Gen Psychiatry. 2002; 59:250-261. ADDITIONAL CITATIONS
Abdeen M A, Stuchley M A: Modelling of magnetic field stimulation of bent neurons. IEEE Trans. Biomed. Eng. 41: 1092-1095, 1994
Amassian, V. E., Cracco, R. Q., Maccabee, P. J., Cracco, J. B., & Henry, K. (1995). Some positive effects of transcranial magnetic stimulation. Advances in Neurology, 67, 79-106.
Barker, A. T., Jalinous, R., & Freeston, I. L. (1985). Non-invasive magnetic stimulation of the human motor cortex. Lancet, 1, 1106-1107.
Bohning D E, Pecheny A P, Epstein C M, Speer A M, Vincent D J, Dannels W R, and George M S, Mapping Transcranial Magnetic Stimulation (TMS) Fields In-vivo with MRI, NeuroReport 8: 2535-2538, (1997).
Bohning D E, Shastri A, McGavin L, McConnell K A, Nahas Z, Lorberbaum J P and George M S, Brain Activity for Transcranial Magnetic Stimulation (TMS) Induced and Volitional Movement are Similar in Location and Level. Investigative Radiology 35 (11): 676-683, 2000.
Bohning, D. E., Shastri, A., Nahas, Z., Lorberbaum, J. P., Anderson, S. W., Dannels, W., Vincent, D. J., & George, M. S. (1998). Echoplanar BOLD fMRI of Brain Activation Induced by Concurrent Transcranial Magnetic Stimulation (TMS). Investigative Radiology, 33 (6), 336-340.
Bohning, D. E. (2000). Introduction and Overview of TMS Physics. In M. S. George & R. H. Belmaker (Eds.). Transcranial Magnetic Stimulation in Neuropsychiatry (pp. 13-44). Washington, D.C.: American Psychiatric Press
Bohning, D. E., Shastri, A., McGavin, L., McConnell, K. A., Nahas, Z., Lorberbaum, J. P., Roberts, D. R., & George, M. S. (2000a). Motor cortex brain activity induced by 1-Hz transcranial magnetic stimulation is similar in location and level to that for volitional movement. Invest Radiol, 35 (11), 676-683.
Bohning, D. E., Shastri, A., Wassermann, E. M., Ziemann, U., Lorberbaum, J. P., Nahas, Z., Lomarev, M. P., & George, M. S. (2000b). BOLD-fMRI response to single-pulse transcranial magnetic stimulation (TMS). Journal of Magnetic Resonance Imaging, 11, 569-574.
Bohning, D. E., He, L., George, M. S., & Epstein, C. M. (2001). Deconvolution of transcranial magnetic stimulation (TMS) maps. Journal of Neural Transmission, 108, 35-52.
Boroojerdi, B., Phipps, M., Kopylev, L., Wharton, C. M., Cohen, L. G., & Grafman, J. (2001). Enhancing analogic reasoning with rTMS over the left prefrontal cortex. Neurology, 56, 526-528.
Brasil-Neto, J. P., McShane, L. M., Fuhr, P., Hallett, M., & Cohen, L. G. (1992). Topographic mapping of the human motor cortex with magnetic stimulation: factors affecting accuracy and reproducibility. Electroencephalo Clin Neuro, 85, 9-16.
Cohen, L. G., Roth, B. J., Nilsson, J., Dang, N., Panizza, M., Bandinelli, S., Friauf, W., & Hallett, M. (1990). Effects of coil design on delivery of focal magnetic stimulation. Technical considerations. Electroencephalo Clin Neuro, 75, 350-357.
George, M. S. & Bohning, D. E. (2000). Measuring brain connectivity with functional imaging and transcranial magnetic stimulation (TMS). In B. Desimone (Ed.). Neuropsychopharmacology. Fifth Generation in Progress
George, M. S. & Belmaker, R. H. (2000). Transcranial Magnetic Stimulation in Neuropsychiatry. Washington, D.C.: American Psychiatric Press.
George, M. S., Lisanby, S. H., & Sackeim, H. A. (1999). Transcranial Magnetic Stimulation: Applications in Neuropsychiatry. Archives of General Psychiatry, 56, 300-311.
Grafman, J. (2000). TMS as a Primary Brain Mapping Tool. In M. S. George & R. H. Belmaker (Eds.). Transcranial Magnetic Stimulation (TMS) in Neuropsychiatry (pp. 115-140). Washington, D.C.: American Psychiatric Press, Inc
Kozel, F. A., Nahas, Z., DeBrux, C., Molloy, M., Lorberbaum, J. P., Bohning, D. E., Risch, S. C., & George, M. S. (2000). How the distance from coil to cortex relates to age, motor threshold and possibly the antidepressant response to repetitive transcranial magnetic stimulation. J Neuropsychiatry Clin Neurosci, 12, 376-384.
Lorberbaum, J. P. & Wassermann, E. M. (2000). Safety Concerns of Transcranial Magnetic Stimulation. In M. S. George & R. H. Belmaker (Eds.). Transcranial Magnetic Stimulation in Neuropsychiatry (pp. 141-162). Washington, D.C.: American Psychiatric Press
Nahas, Z., DeBrux, C., Lorberbaum, J. P., Speer, A. M., Molloy, M. A., Liberatos, C., Risch, S. C., & George, M. S. (2000a). Safety of rTMS: MRI scans before and after 2 weeks of daily left prefrontal rTMS for the treatment of depression. Journal of ECT, 16, 380-390.
Nahas, Z., Lomarev, M., Roberts, D. R., Shastri, A., Lorberbaum, J. P., Vincent, D. J., Teneback, C., McConnell, K., Li, X., Bohning, D. E., & George, M. S. (2000b). Left prefrontal Transcranial Magnetic Stimulation produces intensity dependent bilateral effects as measured with interleaved BOLD fMRI. Human Brain Mapping, 11 (5), 520. (abstract)
Pridmore, S., Filho, J. A. F., Nahas, Z., Liberatos, C., & George, M. S. (1998). Motor threshold in Transcranial Magnetic Stimulation: A comparison of a neurophysiological and a visualization of movement method. The Journal of ECT, 14, 25-27.
Reilly J P: Peripheral nerve stimulation by induced electric currents: Exposure to time varying magnetic fields. Med. Biol. Eng. Comput. 27: 101-110, 1989
Roth, B. J., Saypol, J. M., Hallett, M., & Cohen, L. G. (1991). A Theoretical calculation of the electric field induced in the cortex during magnetic stimulation. Electroencephalo Clin Neuro, 81, 47-56.
Saypol, J. M., Roth, B. J., Cohen, L. G., & Hallett, M. (1991). A theoretical comparison of electric and magnetic stimulation of the brain. Annals of Biomedical Engineering, 19, 317-328.
Topper, R., Mottaghy, F. M., Brugmann, M., Noth, J., & Huber, W. (1998). Facilitation of picture naming by focal transcranial magnetic stimulation of Wernicke's area. Experimental Brain Research, 121, 371-378.
Wassermann, E. M., Cohen, L. G., Flitman, S. S., Chen, R., & Hallett, M. (1996a). Seizures in Healthy People with Repeated Safe Trains of Transcranial Magnetic Stimuli. Lancet, 347, 825-826.
Wassermann, E. M., Grafman, J., Berry, C., Hollnagel, C., Wild, K., Clark, K., & Hallett, M. (1996b). Use and Safety of a new repetitive transcranial magnetic stimulator. Electroencephalo Clin Neuro, 101, 412-417.
Wassermann, E. M. (1997). Report on risk and safety of repetitive transcranial magnetic stimulation (rTMS): Suggested guidelines from the International Workshop on Risk and Safety of rTMS (June 1996). Electroencephalo Clin Neuro,
Amassian, V. E., Cracco, R. Q., Maccabee, P. J., Cracco, J. B., & Henry, K. (1995). Some positive effects of transcranial magnetic stimulation. Advances in Neurology, 67, 79-106.
Ben-Shakkar G, Dolev K (1996): Psychophysiological detection through the guilty knowledge technique: Effect of mental countermeasures. Journal of Applied Psychology 81: 273-281.
Bohning, D. E. (2000). Introduction and Overview of TMS Physics. In M. S. George & R. H. Belmaker (Eds.). Transcranial Magnetic Stimulation in Neuropsychiatry (pp. 13-44). Washington, D.C.: American Psychiatric Press.
Bohning, D. E. Washington, D.C. Introduction and overview of TMS physics. In Transcranial Magnetic Stimulation in Neuropsychiatry. M. S. George and R. H. Belmaker, editors. American Psychiatric Press, 1999. 13-44.
Bohning, D E, Pecheny, A P, Epstein, C M, et al. Mapping transcranial magnetic stimulation (TMS) fields in vivo with MRI. NeuroReport 1997. 8:2535-2538.
Boroojerdi, B., Phipps, M., Kopylev, L., Wharton, C. M., Cohen, L. G., & Grafman, J. (2001). Enhancing analogic reasoning with rTMS over the left prefrontal cortex. Neurology, 56, 526-528.
Brasil-Neto, J. P., McShane, L. M., Fuhr, P., Hallett, M., & Cohen, L. G. (1992). Topographic mapping of the human motor cortex with magnetic stimulation: factors affecting accuracy and reproducibility. Electroencephalo Clin Neuro, 85, 9-16.
Brett A S, Phillips M, Beary F J m (1986): Predictive Power of the Polygraph: Can the “Lie Detector” Really Detect Liars? Lancet I (no. 8480), 544.
Bush G, Whalen P J, Rosen B R, Jenike M A, McInerney S C, Rauch S L (1998): The counting Stroop: an interference task specialized for functional neuroimaging—validation study with functional MRI. Human Brain Mapping. 6: 270-282.
Cohen, L. G., Roth, B. J., Nilsson, J., Dang, N., Panizza. M., Bandinelli, S., Friauf, W., & Hallett, M. (1990). Effects of coil design on delivery of focal magnetic stimulation. Technical considerations. Electroencephalo Clin Neuro, 75, 350-357.
Critchley H D, Elliot R, Mathias C J, Dolan R J (2000): Neural Activity Relating to Generation and Representation of Galvanic Skin Conductance Response: A Functional Magnetic Resonance Imaging Study. The Journal of Neuroscience 20: 3033-3040.
Ekman P, O'Sullivan M, Frkiesen W V, Scherer KR (1991): Invited article: Face, voice and body in detecting deceit. Journal of Nonverbal Bahavior; 15: 125-135.
Elliot R, Rubinsztein J S, Sahakian B J, Dolan R J (2000): Selective attention to emotional stimuli in a verbal go/no-go task: an fMRI study. Neuroreport 11: 1739-1744.
Epstein C M, Woodard J L, Stringer A Y, Bakay A E, Henry T R, Pennell P B, Litt B: Repetitive transcranial magnetic stimulation does not replicate the WADA test. Neurology 2000, 55.
Farwell L A, Donchin E (1991): The Truth Will Out: Interrogative Polygraphy (“Lie Detection”) With Brain Evoked Potentials. Psychophysiology 28: 531-547.
Fernandez G, de Greiff A, von Oertzen J, Reuber M, Lun S, Klayer P, Ruhlmann J, Reul J, Elger C E (2001): Language mapping in less than 15 minutes: real-time functional MRI during routine clinical investigation. Neuroimage 14: 585-94.
Furedy J J (1986): Lie Detection as Psychophysiological Differentiation: Some Fine Lines. In: Coles M G, Donchin E, Porges SW, editors. Psychophysiology Systems, Processes and Applications. New York: The Guilford Press, pp. 683-701.
George M S, Ketter T A, Parekh P I, Rosinsky N, Ring H A, Pazzaglia P J, Marangell L, Post R M (1997): Blunted Left Cingulate Activation in mood Disorder Subjects During a Response Interference Task (The Stroop). Journal of Neuropsychiatry and Clinical Neurosciences 9: 55-63.
Honts C R, Raskin D C, Kircher J C (1987): Effects of physical countermeasures and their electromyographic detection during polygraph tests for deception. Journal of Psychophysiology; 1:21-247.
Kozel, F. A., Nahas, Z., DeBrux, C., Molloy, M., Lorberbaum, J. P., Bohning, D. E., Risch, S. C., & George, M. S. (2000). How the distance from coil to cortex relates to age, motor threshold and possibly the antidepressant response to repetitive transcranial magnetic stimulation. J Neuropsychiatry Clin Neurosci, 12, 376-384.
Kozel F A, Revell L, Lorberbaum J P, Shastri A, Nahas Z, Horner M D, Vincent D J, Lomarev M, Bohning D E, George M S: Brain Regions Involved in Deception; an fMRI study in healthy volunteers. Journal of Neuropsychiatry and Clinical Neurosciences 2001, 13; P63: p. 147. (Abstract)
Lancaster, J L, Rainey L H, Summerlin J L, Freitas C S, Fox P T. Automated labeling of the human brain: A preliminary report on the development and evaluation of a forward-transform method. Human Brain Mapping. 1997; 5:238-242.
Lorderbaum J P, Newman J D, Dubno J R, Horwitz A R, Nahas Z, Teneback C C, et al. (1999): Feasibility of using fMRI to study mothers responding to infant cries. Depression and Anxiety, 10: 99-104.
Lubow R E, Fein 0 (1996): Pupillary size in response to a visual guilty knowledge test: New technique for the detection of deception. Journal of Experimental Psychology: Applied 2: 164-177.
Lykken D T (1998): A tremor in the blood: Use and abuse of the lie detector. New York: Plenum Press.
MacDonald A W III, Cohen J D, Stenger V A, Cartr C S (2000). Dissociating the Role of the Dorsolateral Prefrontal and Anterior Cingulate Cortex in Cognitive Control. Science 288: 1835-1838.
Nahas Ziad, Teneback Charlotte C, Kozel Andy, Speer Andrew M., DeBrux Cart, Molloy Monica, Stallings Lauri, Spicer Kenneth M, Arana George, Bohning Daryl E, George Mark S: Brain Effects of Transcranial Magnetic Stimulation Delivered Over Prefrontal Cortex in Depressed Adults: The Role of Stimulation Frequency and Distance from Coil to Cortex. The Journal of Neuropsychiatry and Clinical Neurosciences 2001; 13: 459-470.
O'Doherty J, Kringelbach M L, Rolls E T, Hornak J, Andrews C (2001): Abstract reward and punishment representations in the human cortex. Nature Neuroscience 4: 95-102.
Pardo J V, Fox P T, Raichle M E (1991): Localization of a human system for sustained attention by positron emission tomography. Nature 349: 61-64.
Pavlidis I, Eberhardt N L, Levine J A (2002). Seeing through the face of deception: Thermal imaging offers a promising hands-off approach to mass security screening. Nature 415: 35.
Pridmore, S., Filho, J. A. F., Nahas, Z., Liberatos, C., & George, M. S. (1998). Motor threshold in Transcranial Magnetic Stimulation: A comparison of a neurophysiological and a visualization of movement method. The Journal of ECT, 14, 25-27.
Rauch S L, Savage C R (1997): Neuropsychiatry of the Basal Ganglia: Neuroimaging and Neuropsychology of the Striatum. Psychiatric Clinics of North America 20: 741-768.
Roth, B. J., Saypol, J. M., Hallett, M., & Cohen, L. G. (1991). A Theoretical calculation of the electric field induced in the cortex during magnetic stimulation. Electroencephalo Clin Neuro, 81, 47-56.
Saypol, J. M., Roth, B. J., Cohen, L. G., & Hallett, M. (1991). A theoretical comparison of electric and magnetic stimulation of the brain. Annals of Biomedical Engineering, 19, 317-328.
Shastri A, Lomarev M P, Nelson S J, George M S, Holzwarth M R, Bohning D E (2001): A low-cost System for Monitoring Skin Conductance During Functional MRI. Journal of Magnetic Resonance Imaging 14: 187-193.
Sheehan P W, Statham D (1988): Associations between lying and hypnosis: An empirical analysis. British Journal of Experimental & Clinical Hypnosis 5: 87-94.
Sporer S L (1997): The less traveled road to truth: Verbal cues in deception detection in accounts of fabricated and self-experiences events. Applied Cognitive Psychology 11: 373-397.
Steinbrook R (1992): The polygraph test: A flawed diagnostic method. New England Journal of Medicine 327: 122-123.
Talairach J, Tournoux P. Co-Planar Stereotaxic Atlas of the Human Brain 3-Dimensional Proportional System: An Approach to Cerebral Imaging. New York, N.Y.: Thieme Medical Publishers, Inc.; 1988.
Topper, R., Mottaghy, F. M., Brugmann, M., Noth, J., & Huber, W. (1998). Facilitation of picture naming by focal transcranial magnetic stimulation of Wernicke's area. Experimental Brain Research, 121, 371-378.

Throughout this application, including the citation lists, various publications have been referenced. The disclosures of these publications in their entireties are hereby incorporated by reference into this application in order to more fully describe the state of the art to which this invention pertains.

While various embodiments of the invention are described above and illustrated in the drawings, it is to be understood that certain changes can be made in the form and arrangement of the elements of each system and steps of each method according to the present invention as would be known to one skilled in the art without departing from the underlying scope of the invention as is particularly described above. Furthermore, the embodiments described above are only intended to illustrate the principles of the present invention and are not intended to limit the invention to the disclosed elements.

What is claimed is:

1. A method for examining cortical sensitivity of a subject, comprising:
applying transcranial magnetic stimulation (TMS) pulses to one or more regions of the brain of a subject;
synchronizing functional brain imaging with the application of the TMS pulses;
determining a blood oxygenation level-dependent (BOLD) response of one or more brain regions to the application of the TMS pulses based on the images produced by the functional brain imaging; and
determining cortical sensitivity over substantially the entire brain cortex of the subject based on the BOLD response.

2. The method of claim 1, wherein the functional brain imaging is performed using functional magnetic resonance imaging.

3. The method of claim 1, wherein the TMS pulses are electrical impulses separated by a variable interstimulus interval (ISI).

4. The method of claim 3, wherein the step of determining a BOLD response includes using a modulation of the BOLD response amplitude as a function of the ISI between pairs of TMS pulses to test intracortical inhibition and facilitation over the entire brain cortex.

5. A method for examining brain communication in a subject, comprising:
applying transcranial magnetic stimulation (TMS) pulses to stimulate one or more regions of the brain of the subject;
synchronizing functional brain mapping with the application of the TMS pulses;
determining a blood oxygenation level-dependent (BOLD) response of one or more brain regions to the application of the TMS pulses based on the images produced by the functional brain imaging; and
examining brain communication based on the BOLD response.

6. The method of claim 5, wherein the function brain imaging is performed using functional magnetic resonance imaging (fMRI).

7. The method of claim 5, wherein the TMS pulses are electrical impulses seperated by a variable interstimulus interval (ISI).

8. The method of claim 7, wherein the step of determining the BOLD response includes using a modulation of the BOLD response amplitude as a function of the ISI between pairs of TMS pulses to examine brain communication at high time resolution.

9. The method of claim 8, wherein brain communication is examined at time resolutions an order of magnitude greater than that of the hemodynamic response of the subject.

10. A system for examining cortical sensitivity of a subject, comprising:
means for applying transcranial magnetic stimulation (TMS) pulses to one or more regions of the brain of a subject;
means for synchronizing functional brain imaging with the application of the TMS pulses;
means for determining a blood oxygenation level-dependent (BOLD) response of one or more brain regions to the TMS pulses based on the images produced by the functional brain imaging; and
means for examining cortical sensitivity over the entire brain cortex based on the BOLD response.

11. The system of claim 10, wherein the functional brain imaging is performed using functional magnetic resonance imaging (fMRI).

12. The system of claim 10, wherein the TMS pulses include electrical impulses seperated by a variable interstimulus interval (ISI).

13. The system of claim 12, wherein a modulation of the BOLD response amplitude as a function of the ISI between pairs of TMS pulses is used to examine intracortical inhibition and facilitation over substantially the entire brain cortex of the subject.

14. A system for examining brain communication, comprising:
means for applying transcranial magnetic stimulation (TMS) pulses to stimulate one or more regions of the brain of the subject;
means for synchronizing functional brain imaging with the application of the TMS pulses;
means for determining a blood oxygenation level-dependent (BOLD) response of one or more brain regions to based on the images produced by the functional brain imaging; and
means for examining brain communication based on the BOLD response.

15. The system of claim 14, wherein the functional brain imaging is performed using functional magnetic resonance imaging.

16. The system of claim 14, wherein the TMS pulses include electrical impulses seperated by a variable interstimulus interval (ISI).

17. The system of claim 16, wherein a modulation of the BOLD response amplitude as a function of the ISI between pairs of TMS pulses is used to examine brain communication at high time resolution.

18. The system of claim 17, wherein brain communication is examined at time resolutions an order of magnitude greater than that of the hemodynamic response.

19. A method for examining medication effects on the brain of a subject, comprising:
applying transcranial magnetic stimulation (TMS) pulses to one or more regions of the brain of the subject to which medications have been given;
synchronizing functional brain imaging with the application of the TMS pulses; and
examining medication effects on the brain based on the synchronized images produced by the functional brain imaging.

20. The method of claim 19, wherein the functional brain imaging is performed using functional magnetic resonance imaging.

21. The method of claim 19, wherein the step of examining medication effects includes determining a blood oxygenation level-dependent (BOLD) response of one or more brain regions to the TMS pulses based on the images produced by the functional brain imaging.

22. The method of claim 21, wherein the step of examining includes examining effects of the medication on a resting motor threshold of the patient.

23. The method of claim 19, wherein the TMS pulses are applied to at least one of motor cortex and the prefrontal cortex of the brain of the subject.

24. The method of claim 19, wherein the medication includes at least one of a central nervous system (CNS) active compound and a placebo.

25. The method of claim 24, wherein the medication includes lamotrigine (LTG).

26. A system for examining effects of medication on the brain of a subject, comprising:
means for applying transcranial magnetic stimulation (TMS) pulses over one or more regions of the brain of a subject to which medication has been given;
synchronizing functional brain imaging with the application of the TMS pulses to produce images; and
examining effects of medication on the one or more regions of the brain based on the synchronized images produced by the functional brain imaging.

27. The system of claim 26, wherein the functional brain imaging is performed using functional magnetic resonance imaging (fMRI).

28. The system of claim 26, wherein the step of examining effects includes determining a blood oxygenation level-dependent (BOLD) response of the one or more brain regions to the application of the TMS pulses based on the images produced by the functional brain imaging.

29. The system of claim 26, wherein the step of examining includes examining resting motor threshold of the subject.

30. The system of claim 26, wherein the TMS is applied to at least one of the motor cortex and the prefrontal cortex of the brain.

31. The system of claim 26, wherein the medication includes at least one of a central nervous system (CNS) active compound and a placebo.

32. The system of claim 31, wherein the medication includes LTG.
